(19) United States
(12) Patent Application Publication
(10) Pub. No.: US 2024/0279727 A1
(43) Pub. Date: Aug. 22, 2024

(54) DEVICE AND METHOD FOR PROCESSING BIOLOGICAL SAMPLES

(71) Applicant: STAR ARRAY PTE. LTD., Singapore (SG)

(72) Inventor: Haiqing GONG, Singapore (SG)

(73) Assignee: STAR ARRAY PTE. LTD., Singapore (SG)

(21) Appl. No.: 18/641,454

(22) Filed: Apr. 22, 2024

Related U.S. Application Data
(63) Continuation-in-part of application No. PCT/SG2022/050748, filed on Oct. 20, 2022.

(30) Foreign Application Priority Data
Oct. 21, 2021 (SG) 10202111690U
May 18, 2022 (SG) 10202205151V

(51) Int. Cl. C12Q 1/6848; C12Q 1/6806; G01N 35/02; G01N 35/10

(52) U.S. Cl. CPC C12Q 1/6848; C12Q 1/6806; G01N 35/026; G01N 35/1067; B01L 2200/04; B01L 2200/141; B01L 2200/16

(57) ABSTRACT

Cartridge module, apparatus and method are provided for improving contamination control during biological analyses or nucleic acid analyses protocol. The cartridge module includes a hollow tube and receptacles. A cover layer isolates the receptacles from the ambience while the receptacles allow the tube when attached to a syringe to dispense and aspirate a reagent liquid or a biological sample to and from the receptacle through the cover layer only from a receptacle top opening. The cover layer substantially blocks aerosol exchange between the ambience and the receptacle during the whole process of analyses. Ambience contamination due to accidental spillage from the receptacles is also prevented. A connecting air space reduces change of air pressure inside the receptacles during aspiration or dispensation of liquid.

### CROSS REFERENCE TO THE RELATED APPLICATIONS

This application is a continuation-in-part application of International Application No. PCT/SG2022/050748, filed on Oct. 20, 2022, which is based upon and claims priority to Singapore Patent Applications No. 10202111690U, filed on Oct. 21, 2021, and No. 10202205151V, filed on May 18, 2022, the entire contents of which are incorporated herein by reference.

### TECHNICAL FIELD

The present invention relates to the device and method for processing biological samples, and particularly for performing extraction, amplification reaction and detection of nucleic acid.

### BACKGROUND

Extraction and amplification of nucleic acids is increasingly important to molecular biology, food safety and environmental monitoring. A large number of biological researchers use polymerase chain reaction (PCR) in their work on nucleic acid analyses, due to its high sensitivity and specificity. The PCR is typically conducted by thermal cycling process that is adapted to heat and cool receptacles containing the reaction material to different temperatures for DNA denaturation, annealing and extension. In a real time PCR assay, a positive reaction is detected by accumulation of a fluorescent signal. Fluorescence imaging is typically conducted multiple times or after every thermal cycle to record the progressive change in the biological sample, and the amount of the target nucleic acid in the sample or biological sample is detected. PCR amplification of minute quantities of DNA is conducted for applications like research, forensic analyses, wildlife studies, ultrasensitive diagnostics and the kind. Isothermal amplification is also used. Typically, the PCR is preceded by steps of sample preparation such as nucleic acid extraction and purification, in which multiple steps of liquid handling are performed, including liquid aspiration, dispensing, and mixing. Both sample preparation steps and the nucleic acid amplification steps may cause contamination, mainly due to aerosol and/or vapor generation. Typical approach to prevent the nucleic acid analyzer or sample processing instrument from contaminating a lab space is to seal the PCR receptacles during an amplification process and put the sample preparation instrument in a biological safety cabinet or a hood having negative pressure where the aerosol and/or vapor generated from liquid handling is removed from the biological safety cabinet. Typically, to prevent said contamination in a nucleic testing lab, sample preparation for nucleic acid extraction and PCR steps are performed in different rooms with controlled air pressure and air flow direction. Use of such a biological safety cabinet or a hood and a controlled testing environment is not technologically suitable or cost effective for nucleic acid testing to be performed outside a PCR lab, such as in distributed point of care testing (POCT) sites such as school, pharmacy, airport, or a commercial building.

Nested PCR with a pre-amplification step is a technique that reduces nonspecific amplification of the DNA template, typically with the use of two primer sets or reagents and two successive PCR reactions. The first set of primers are used in an initial PCR reaction. Amplicons resulting from the first PCR reaction are used as template for a second set of primers and a second amplification step. However, the potential for contamination of a lab environment is typically also increased due to opening of the cap of the PCR receptacles used in the first round of amplification to carry out additional liquid transfer and processing of the amplicon products from the first round of amplification in order to prepare for the second round of amplification. To minimize such contamination carryover between the first and the second rounds of amplification, different parts of the process need to be physically separated from one another and conducted preferably in entirely separate rooms. Amplicons from nested PCR assays are detected in the same manner as in PCR. As such, the nested PCR process is hard to be automated in one instrument, due to a large chance of contamination when all steps are performed within an instrument box.

Prior to the nucleic acid amplification step, the extraction and amplification processes for the nucleic acid are often hampered by contamination of the sample from the laboratory surface, reagents, carry over pipettes and pipette tips, lab coats, glove boxes, and waste baskets. The aerosol and/or vapors generated from the samples and reagent liquids contaminate the surrounding ambience. Higher the sensitivity of the assays, more prone are they to the effects of contamination. Successful aerosol contamination control is the backbone of numerous experiments—whether for genotyping, creating NGS libraries, studying single cells, or testing sensitive clinical samples. In the lab, using unsealed wellplates for extraction steps is convenient and provides flexibility for many test protocols. It also has low cost and is easy to have a liquid handling access to the liquids. But, in use, a few separated rooms are needed for the steps of reagent preparation, sample preparation, PCR and the like to control aerosol and/or vapor along with other contaminations. Such facilities are provided in laboratories having trained personnel with air controlling facilities and protocol. Such laboratories generally also have decontamination protocol when contamination occurs. In POCT settings, there are no separated rooms, contamination facilities or trained personnel provided.

Even trace amounts of contamination by the biological samples can have serious negative impact particularly when these get amplified during the amplification process and subsequently cause erroneous detection and diagnosis. Thus both, contamination of the ambience by the biological sample and contamination of the sample by the ambience need to be arrested.

Microfluidic cartridge modules are developed to provide sealed environments, containing internal microchannels, valves, and pumps or pump interfaces requiring complex machine interface to operate the cartridge module. The BioFire FilmArray System provides a new standard for syndromic infectious disease molecular diagnostics, with integrated sample preparation, amplification, detection, and analysis. Patent Publication No: US2018/0214864 A1 and the product GeneXpert describes use of reaction cartridge modules. The disposable cartridge modules constitute micro-valves and micro-pumps for fluid transfer from chamber to chamber. The thermal cycling for the amplification and the real time optical detection are conducted within the chamber itself. However, such cartridge modules are complex to manufacture due to the complex design, hence are expensive. The high complexity also may potentially lead to low performance reliability and be demanding to manufacture with a good production yield. The microfluid cartridge modules are not flexible to be used for different protocols. Number of samples to be evaluated are limited by the design of the cartridge module. Besides, the cartridge module design does not control cross-contamination between the sample and the ambience during the sample preparation and the PCR.

Therefore, there is a large demand to develop simple and low-cost liquid handling device and method to run complicated sample preparation steps for nucleic acid extraction and for PCR without aerosol and/or vapor contamination. Likewise, biological analyses with infectious or foul-smelling samples like faeces is also a challenge with respect to contamination control, particularly in POCT setting

The present invention provides several improvements towards device, apparatus and method for sample preparation, amplification, detection, and analysis. This invention provides a great positive impact on biological analysis by making the disposables much more affordable and reliable due to their simplistic design and functionality. The invention potentially makes the process of amplification faster, provides a notable contamination control particularly for POCT settings and also addresses the issues with biological analyses for infectious or foul-smelling samples like faeces.

### SUMMARY

Unless specified otherwise, the term “comprising” and “comprise” and grammatical variants thereof, are intended to represent “open” or “inclusive” language such that they include recited elements but also permit inclusion of additional, unrecited elements. The word “substantially” does not exclude completely. The term biological sample has been used for the biological sample containing nucleic acid, whether before or after mixing a reagent liquid. The biological sample may originate in any biological form such as swab, saliva, blood and the kind. The reagent liquid may refer to any kind used in the art, such as PCR reagent, extraction liquid, purification liquid, deamination liquid, desulphonation liquid and the kind, which are used for processing of the biological sample. In this disclosure, the receptacles may be in the integrated form as wellplates, or as in disintegrated form.

According to a first aspect, a cartridge module is provided for improving contamination control during biological analyses or nucleic acid analyses protocol including nucleic acid extraction, nucleic acid amplification and detection. The cartridge module comprises: at least one hollow tube; and a plurality of receptacles; at least one cover layer to isolate the receptacles from the ambience outside the cover layer, wherein each of the receptacles allow the tube when attached to a syringe or a pipette in use to dispense and aspirate an in use reagent liquid or in use biological sample to and from the receptacle through the cover layer only from a receptacle top opening, the cover layer is to substantially block aerosol exchange between the ambience and the receptacle during the whole process of biological analyses or nucleic acid analyses even after the tube pierces the cover layer and retracts, the cover layer also prevents contamination of the ambience due to any spillage of the reagent liquid or the biological sample from the receptacles or the cartridge module, the cover layer being attached to: 1) at least one of the receptacle top openings, or 2) the cartridge module or the receptacles while maintaining a connecting air space being isolated from the ambience of the cartridge by the cover layer between the cover layer and the receptacle top openings, the connecting air space facilitating a reduction in change of air pressure inside the isolated receptacles during aspiration or dispensation of the biological sample and the reagent liquid by the tube. The cover layer advantageously scrubs off a significant portion of the reagent liquid or the biological sample adhering on the tube underneath the cover layer while the tube is retracted out of the receptacle. The tube preferably has a bevelled tip. The outer diameter of the tube is between 0.1 mm and 5 mm. The cover layer has a thickness range from 0.1 mm to 50 mm.

According to an embodiment, at least a portion of the cover layer material is porous when the cover layer is attached to the receptacle top openings. The porous cover layer helps in regulating pressure during liquid dispensation and aspiration. The increased or decreased air pressures inside the receptacle also affect the volumes of the reagent liquid or the biological sample that are programmed to be dispensed or aspirated from and into the tube. These effects may impact in small liquid volumes but are significant when the receptacle volumes are also small. More issues are described under FIGS. 7A and 7B. The porous cover layer should have porosity, tortuosity and internal pore structure to substantially block aerosol generated in nucleic acid analysis in a typical laboratory practice.

According to an embodiment, at least a portion of the cover layer material is elastomeric. The elastomeric cover layer helps in substantially closing the cover layer aperture created in the cover layer due to the piercing of the tube.

The material of the cover layer is any one or a combination of the following: a) porous elastomeric, b) porous non-elastomeric, c) non-porous elastomeric, and d) non-porous non-elastomeric. The material of the cover layer can be selected from the four types as described above to optimize between the pressure regulation, blocking of aerosols and preventing the spillage.

According to an embodiment, a cover liquid is pre-dispensed or provided to be dispensed on the cover layer at least where the tube pierces, to form a stack or be soaked by the cover layer or be sandwiched within the cover layer, the cover liquid being non-reacting with the reagent liquids and the biological sample. The cover liquid may also be underside of the cover layer. Advantageously, the cover liquid is suitable for enhancing the isolation of the cover layer by blocking a cover layer aperture caused by the piercing of the cover layer by the tube, particularly when the cover layer is non-elastomeric. The isolation can be hermetic if the cover layer is non-porous. The cover liquid also improves the aerosol blockage as well as the spillage, particularly for porous cover layers. With the cover liquid, particularly for the non-elastomeric cover layer, the receptacle could be maintained hermetically isolated from the ambience before and after piercing of the cover layer by the tube, such as for the steps involving no high temperature. The cover liquid closes the cover layer aperture of non-elastomeric cover layer. Trace amounts of the reagent liquid or the biological sample remaining on the tube get covered by the cover liquid as the tube passes through the cover liquid. This prevents the reagent liquid or the biological sample adhering to the tube from being exposed to the ambience and contaminating the same. The cover liquid also covers a portion of the tube that is inserted within the receptacle, hence entry of any contamination from the ambient into the receptacle is also reduced. When the cover liquid is under the cover layer the cover liquid is also better protected from physical and handling damages. The cover liquid is preferably immiscible with the reagent liquids and the biological sample and is wetting over the tube and the cover layer. The cover liquid may be a viscous liquid polymer or monomer or an oil or liquid wax or any other suitable material in the art.

It will be appreciated by those skilled in the art that the receptacles to undergo amplifications by heat treatment need to be provided with a separate cover layer, to enable heating and transferring the receptacles to an amplification module outside the cartridge.

The claimed features have several advantages as described hereafter: 1) With the isolation, once the reagent liquid and the biological sample are dispensed in the receptacles, these liquids are never exposed to the ambience, right through the processes of biological analyses or nucleic acid extraction, amplification, imaging and disposal. Hence, the contamination of the biological sample from the ambience and vice versa is significantly reduced in comparison to the state-of-the-art cartridges. The isolation significantly prevents aerosols and/or vapors from escaping or entering the receptacle during the analyses. 2) The feature of having the receptacles with provision for liquid communication only from the receptacle top openings, enables the cartridge module to operate with lesser complexity. Thus the cartridge module, in the absence of any valve, thermal cycling module or optical detection module, lowers the cost and enhances the reliability of the detection and analysis. Also, cross-contamination between the biological samples in the receptacles is reduced as there is no provision for liquid communication between the receptacles. This feature also allows customized computer programming of using the receptacles as against the microchannels. 3) In addition, the low cost allows the cartridge module to be disposed once the reagent liquids are consumed, thereby further reducing the contamination possibilities. 4) The cartridge module allows greater flexibility for the user on the choice of baths in an apparatus for amplification, like for a faster thermal cycling the user may use separate baths maintained at the target temperatures in the apparatus instead of cycling the temperature in a single bath or heat block. 5) The simplistic feature of the cartridge module allows it to be easily portable. 6) The cartridge module allows using commonly used disposables in the industry like the individual receptacles and the wellplates. 7) The cover layer isolation also addresses the issues with biological analyses in POCT settings with infectious or foul-smelling samples like faeces.

According to an embodiment, the cartridge further comprises a scrubbing layer that is fixed over the stack such that the cover layer, the cover liquid and the scrubbing layer form a composite layer, the scrubbing layer being pierceable by the tube and substantially scrubs off the excess cover liquid sticking to the tube when being retracted out of the receptacle. Without the scrubbing layer, the cover liquid may get depleted faster when in small quantity or when the tube is long.

According to an embodiment, the cartridge further comprises at least one syringe, providing at least one feature from the group consisting: a) one o-ring between a plunger and a barrel of the syringe, a cover liquid being provided above the o-ring and away from a dispensing or aspirating nozzle of the barrel, and b) at least two o-rings between a plunger and a barrel of the syringe, a cover liquid being provided in between the two o-rings, the cover liquid being immiscible and non-reacting with the reagent liquids and the biological sample. The o-ring with the cover liquid tends to make the syringe better airtight, so that any potential contamination within the receptacle from the ambience and vice versa is further reduced. Besides, the trace amounts of the reagent liquid or the biological sample in the syringe sticking along the inner wall of the plunger and the outer wall of the barrel get covered with the cover liquid when the plunger is pushed downwards into the barrel. This covering helps the reagent liquid or the biological sample from getting exposed to the ambience and contaminating the latter.

According to an embodiment, the cartridge further comprises a rigid holding layer as positioned over or under the cover layer, the rigid holding layer having holding layer apertures aligned over the receptacle top openings for the tube to pierce the cover layer. Advantageously, the rigid holding layer prevents the cover layer from being undesirably detached or flexed when the tube is pierced into or retracted from the receptacle.

According to an embodiment, the cartridge further comprises at least one receptacle holder for holding at least one receptacle, the receptacle holder being detachable from the cartridge. The receptacle holder is useful for carrying the receptacles in and out of the cartridge module, as within the modules of an apparatus for biological analyses or nucleic acid analyses protocol including nucleic acid extraction, nucleic acid amplification and detection.

According to an embodiment, the cartridge further comprises a liquid sealant which when dispensed over at least one of the receptacle top openings can seal the receptacle top openings whether before or after curing, the liquid sealant being immiscible and non-reacting with the reagent liquids and the biological sample. The liquid sealant is dispensable by a tube passed through the cover layer or the stack thereby creating an air gap in the receptacle between the biological sample or the reagent liquid. The liquid sealant may be a viscous one which provides a large resistance to the pressurized air in the receptacle to leak out of the liquid sealant. The sealing helps to prevent aerosols and/or vapors from the biological sample inside the receptacle from emerging out through the cover layer aperture or the stack during the step of amplification where the receptacle is subjected to heat. Any such contamination from the ambience reaching the receptacles is also controlled. Due to the air gap, during imaging fluorescence from the liquid sealant is not unwantedly captured along with that from the biological sample. The liquid sealant may be any viscous material such as wax or glue. Wax that solidifies at room temperatures are easier for shipment of the kit. During its aspiration and dispensation by the tube, it can be locally heated to a liquid form.

According to an embodiment, the cover layer is provided with pits to contain the cover liquid, and the pits are interconnected. A tube tip of the hollow tube can then move between the pits while remaining dipped in the cover liquid and without being exposed to the ambience. If the tube tip is exposed to the ambience after aspiration or dispensation of the biological sample containing the nucleic acid, any dripping of the biological sample contaminates the ambience. The unwanted dripping may occur due to various factors like software error or instrumental error. Such a possibility of contamination is avoided in this embodiment as any such dripped biological sample remains trapped within the cover liquid.

According to an embodiment, the cover layer over at least one of the receptacle top openings is flexible and is provided with at least one through aperture or a partially-through aperture to make the piercing by blunt tubes like with the pipettes easier and with lesser force applied. Blunt tubes are preferred particularly when the receptacles are made with deformable material. Sharp tubes have the potential to pierce through the deformable material.

According to an embodiment the cover layer has a recessed top for guiding a tube tip of a tube before the tube tip pierces the cover layer through a base region of the recessed top, the recessed top having slanted sides to provide a tapering towards the base region, the base region being smaller than the receptacle top opening. The tapering surface is preferred to be a hard surface when the tube is in the form of a sharp needle to prevent the needle from penetrating the tapering surface. This provides better tolerance for misalignment of the tube tip of the syringe or the pipette with reference to the small receptacle top opening. This feature is helpful particularly when the receptacles are in the form of very narrow capillaries to increase the efficiency of heat transfer during the amplification of the nucleic acid in the biological sample. The slanted sides are made of a rigid material to prevent piercing by the tube tip.

The cartridge module may include at least one receptacle containing magnetic beads for binding nucleic acids from the biological sample. At least one receptacle may contain oil or water or a DNA removing agent as a cleaning liquid for cleaning the tube and the syringe at any stage during the nucleic acid analyses, including inner and outer surfaces of the needle tube, the liquid retaining space as shown in FIGS. 24A-24B, the joining area where a syringe engages the tube head. These regions have surface geometries that undesirably retain from the previous processing steps. The cleaning also removes the biological samples from previous handlings when a syringe and a tube are used from processing multiple samples.

According to an embodiment of the cartridge module, at least a portion of the receptacles is made of a deformable material, so that in use, (i) the receptacles in an initial condition or shape can bloat to accommodate the dispensed reagent liquid or the biological sample, and (ii) the receptacles in an initial condition or shape can collapse when the dispensed reagent liquid or the biological sample is aspirated out, thereby facilitating a reduction in change of air pressure inside the isolated receptacles during aspiration or dispensation of the biological sample and the reagent liquid by the tube. This embodiment is useful particularly for the non-porous cover layer. The increased or decreased air pressures inside the receptacle also affect the volumes of the reagent liquid or the biological sample that are programmed to be dispensed or aspirated from and into the tube. These effects may impact in small liquid volumes but are significant when the receptacle volumes are also small.

According to an embodiment, the cartridge module with the connecting air space further comprises: a sealing layer sealing the receptacle top openings individually or in plurality to prevent any spillage of the reagent liquid and the biological sample from within the receptacles when the cartridge module is not in upright position, the sealing layer being pierceable by the tube. During use vent apertures needs to be generated in the sealing layer by a tube attached to the syringe or pipette mechanism, so that the receptacles gets in air communication with the connecting air space during subsequent aspiration or dispensation from or into the receptacles.

According to an embodiment, when the cover layer is attached to the receptacle top openings, a plurality of the isolated receptacles are vertically positioned in a pile, such that a tube tip of the hollow tube can pierce through the cover layers of each receptacle in the pile to reach a lower most receptacle in the pile from a topmost receptacle in the pile. This embodiment helps to reduce the foot-print of the cartridge module which is desirable for a portable apparatus.

According to an embodiment, the cartridge module further comprises: a fixed pattern that can be detected by an apparatus during auto-positioning of the tube over the receptacle top openings.

According to an embodiment the cartridge module further accommodates at least one sample container with a cap that in use contains the biological sample, the cap at least in part includes the cover layer that can be pierced by the tube to aspirate the biological sample from the sample container and dispense into the receptacle. This improves the contamination control as the biological sample is transferred into the receptacle by the tube while the sample container is in the cartridge module. The cap and the cover layer in the cap prevent any possible spillage of the biological sample during the whole analysis until disposal of the cartridge module.

According to a second aspect, apparatus is provided for improving contamination control during biological analyses or nucleic acid analyses protocol including nucleic acid extraction, nucleic acid amplification and detection. The apparatus comprises: a) receiving module for accommodating the cartridge module according to any one preceding claim; b) robotically controlled syringe or pipette mechanism for operating the hollow tube attached to a syringe or a pipette to aspirate and dispense the biological sample or reagent liquid from and into the receptacles via the receptacle top openings and through the cover layers or the stacks or the sandwich; c) an amplification module to provide isothermal heating or cyclic heating for amplification of the nucleic acid in the receptacle, the amplification being conductible while the receptacle containing the biological sample is within the cartridge module or outside; d) an optical imaging mechanism for imaging of the biological sample during or after the amplification, while the receptacle containing the biological sample is within the cartridge module or outside; and e) a robotically controlled transfer mechanism that is configurable to position the receptacle as held by a receptacle holder as provided in the cartridge module or in the apparatus, to outside the cartridge module, when the amplification is conducted outside the cartridge module.

According to an embodiment, the apparatus is computer programmable to operate the syringe or pipette mechanism to press the tube as attached to the syringe against a fixed surface in the cartridge module or in the apparatus and bend the tube, and thereafter mount the syringe with the bent tube in the cartridge module. The advantage has been described under the first aspect. Advantageously, bending the tube as attached to the syringe or pipette after use enables easier for accommodation in the cartridge before disposal.

The apparatus further comprises an ultraviolet (UV) curing module to cure a UV resin deposited at the receptacle top opening. Such in-situ curing before amplification is helpful to block the vapors from the biological sample in the receptacle to contaminate the ambience through the cover layer aperture formed in the cover layer due to piercing of the tube.

According to an embodiment, the apparatus is further computer programmable to position the receptacle holder or the cartridge module such that during aspiration and dispensation of the reagent liquid and the biological sample by the syringe or pipette operating mechanism from and into the plurality of receptacles, the portion of the tube that contacted the biological sample in the receptacle does not move out of the connecting air space. This reduces the contamination significantly.

According to an embodiment, the apparatus is computer programmable to conduct at least two steps of amplifications with treatment of the biological sample by the reagent liquid between any two consecutive steps of amplifications and through the cover layer with or without the cover liquid, while the receptacle containing the biological sample remains within the cartridge module or outside but remains within the apparatus. The invention enables nested dual amplification of the nucleic acid in the biological sample to be carried out automatically within the same receptacle with the cover layer or the stack. The PCR amplicons need not be taken out of the receptacle after one amplification, for mixing with the reagent liquid before another amplification. The method is thus suitable for a fast point of care testing (POCT) PCR process.

This product automates the highly complex and time-consuming manual procedures, with minimal chances for contamination. More than two amplifications may be desirable to improve the sensitivity of detection of the DNA and RNA under investigation.

According to an embodiment, the apparatus further comprises: a detection module to detect misalignment of a tube tip with respect to the receptacle top openings due to piercing through the cover layer; and an auto-calibrating module for the syringe or pipette mechanism to compensate the misalignment. The extent of the misalignment suffered by the tube tip depends on the material of the cover layer, its thickness and the kind. Thinner the tube, more susceptible it is to bending during making, assembly onto the cartridge module and during use where it subjected to forces during piercing the cover layer. Therefore, the tube may be unable to enter the receptacle through the receptacle top opening such as for PCR or in a glass capillary. The tube may also be unable to enter a desirable area of a receptable during magnetic extraction and avoid hitting the magnetic beads clump. One way to find the tube tip position before and during the operation of the cartridge module is that the tube tip approaches a fixed pattern on the cartridge module, and a camera or a sensor records image or signals of the tube tip and the pattern and calculates the coordinates of the tube tip in reference to a position of the pattern. The calculated coordinates are sent to an instrument controller in the apparatus to generate a motion command to move the tube tip to a target position required in a test protocol. The sensor may be optical or electrical or magnetic.

According to an embodiment, the apparatus the detection module optically detects misalignment of the tube tip with respect to a fixed pattern on the cartridge module.

According to an embodiment, the apparatus further comprises: a syringe pickup module to pick up at least one syringe from the cartridge module.

According to an embodiment, the apparatus further comprises: a tube pick up module for mounting the tube as provided in the cartridge module onto a syringe.

According to an embodiment, the apparatus further comprising: a positioning module to detect a fixed pattern on the cartridge module and position the tube over the receptacle top openings when the tube is attached to the syringe or pipette mechanism. Before and during the operation of the cartridge module the tube tip approaches a fixed pattern on the cartridge module, and a camera or a sensor records image or signals of the tube tip and the pattern and calculates the coordinates of the tube tip in reference to a position of the pattern. The calculated coordinates are sent to an instrument controller in the apparatus to generate a motion command to move the tube tip to a target position required in a test protocol. The sensor may be optical or electrical or magnetic.

According to an embodiment, the apparatus further accommodates at least one sample container with a cap that in use contains the biological sample, the cap at least in part includes the cover layer, the syringe or pipette mechanism being computer programmable to aspirate the biological sample from the sample container by piercing the tube through the cover layer and dispense into the receptacle. This improves the contamination control as the biological sample is transferred into the receptacle by the tube while the sample container is in the cartridge module. The cap and the cover layer in the cap prevent any possible spillage of the biological sample during the whole analysis until disposal of the cartridge module.

According to a third aspect, a method is provided for improving contamination control during biological analyses and nucleic acid analyses protocol including nucleic acid extraction, nucleic acid amplification and nucleic acid detection, the method employing any of the cartridge modules and the apparatus as described above; loading the cartridge module with the reagent liquids and biological sample in the receptacles into the receiving module; conducting biological analyses or nucleic acid analysis within the apparatus without exposing the reagent liquid and biological sample to the ambience, by aspirating and dispensing the reagent liquid and the biological sample from and into the receptacles by piercing and retracting the tube through the cover layer; and disposing the cartridge module with the receptacles with the cover layer, reagent liquids, the biological sample and the tube. The advantages have been described under the second aspect.

According to an embodiment, the method comprises: operating the syringe or pipette operating mechanism according to one step from the group consisting: a) after dispensing the reagent liquid or the biological sample in the receptacle, when a tube tip of the tube has reached above the reagent liquid or the biological sample in the receptacle, conducting the syringe or the pipette in a suction mode for a predetermined time to release at least a part of an excess air pressure in the hermetically isolated receptacle that is created due to the dispensing, b) while the tube is inserted into the receptacle and before aspirating, with a tube tip of the tube being above the reagent liquid or the biological sample in the receptacle, conducting the syringe or the pipette in an ejection mode for a predetermined time to increase an air pressure in the hermetically isolated receptacle for at least partially compensating a vacuum to be created by the aspiration of the reagent liquid or the biological sample, and c) while the tube is taken out of the receptacle when a tube tip of the tube has reached the cover liquid in the stack, holding the tube for a predetermined time to release within the cover liquid a portion of the reagent liquid or the biological sample adhering to the tube. This feature reduces air pressure variation during liquid handling from and to the receptacles during liquid aspiration and dispensing. The air pressure inside the receptacle is increased due to dispensation by the tube. When the tube is thereafter retracted out of the hermetically isolating cover layer, the reagent liquid or the biological sample drips under the atmospheric pressure, thereby exposing the biological sample or the reagent liquid to the ambience. Similarly, the air pressure inside the receptacle is decreased due to aspiration by the tube. When the tube is thereafter retracted out of the hermetically isolating cover layer, the ambient air forces into the tube thereby causing chances for contamination from the ambience. The increased or decreased air pressures inside the receptacle also affect the volumes of the reagent liquid or the biological sample that are programmed to be dispensed or aspirated from and into the tube. These effects may impact in small liquid volumes but are significant when the receptacle volumes are also small.

According to an embodiment the method further comprises: dispensing the cover liquid as provided in at least one of the receptacles, on selected regions of the cover layer while the receptacle is in the cartridge module. The dispenser may be in any form in the art. Dispensing the cover liquid on the cover layer outside or inside the apparatus just before use is helpful in protecting the cover liquid from getting displaced or wiped off during transport or handling.

According to an embodiment of the method, employing the tube being attached to a pipette containing a filter and a liquid layer in the pipette, the filter and the liquid layer being separated by a first gap in between and the liquid layer being closer to a tube tip; and operating the syringe or pipette operating mechanism to maintain a second gap between the liquid layer and the reagent liquid or the biological sample as aspirated. The filter and the liquid layer block the vapors or aerosols from the biological sample to enter the pipette only to a limited extent thereby reducing contamination of the apparatus.

According to an embodiment the method comprises adapting to either step according to the group consisting: a) injecting a liquid sealant through the cover layer to seal the receptacle top opening before amplification of the biological sample in the receptacle, and b) disposing a liquid sealant on the cover layer over the receptacle top opening or on the stack, before amplification of the biological sample inside the receptacle. The liquid sealant may be viscous enough to provide a large resistance to the pressurized aerosols and/or vapors in the receptacle to leak out of the cover layer aperture or the stack during the step of amplification where the receptacle is subjected to heat. The liquid sealant may be cured by any means in the art. Herein, the receptacle top opening region is narrow enough to provide enough surface tension for holding the liquid sealant. The liquid sealant forms an air gap over the biological sample so that during imaging, fluorescence from the liquid sealant is not unwantedly captured along with that from the biological sample. The liquid sealant may be wax or glue. Wax that solidifies at room temperatures are easier for shipment of the cartridge module. During its aspiration and dispensation by the tube, it can be locally heated to a liquid form.

According to an embodiment the method comprises computer programming the syringe or pipette mechanism for the tube to pierce the cover layer or the stack at selected locations of the cover layer over the receptacle top openings, such that a size of a cover layer aperture created on the cover layer is minimized during multiple piercings.

According to an embodiment, at least once during analyses, the method further comprises: cleaning the tube and the syringe by aspirating and dispensing a cleaning liquid by the tube.

According to an embodiment, the method comprises: employing the cartridge module with the sealing layer; and generating a vent aperture in the sealing layer over the receptacle top opening by the tube attached to the syringe or pipette mechanism, so that the receptacle is in air communication with the connecting air space during subsequent aspiration or dispensation from or into the receptacle.

According to an embodiment, the method comprises: employing the apparatus to detect misalignment of the tube with respect to the receptacle top openings due to piercing through the cover layer; and calibrating the syringe or pipette mechanism to compensate the misalignment.

According to an embodiment, the method comprises: at least once, aspirating a filler liquid in the syringe by the tube; and dispensing the filler liquid, such that the filler liquid: a) fills any existing liquid retaining space in the tube head that could otherwise retain the reagent liquid and the biological sample during the subsequent dispensation after aspiration, or b) replaces the reagent liquid and the biological sample pre-occupying the liquid retaining space from previous processing, the filler liquid being immiscible with, heavier than and non-reacting with the reagent liquid and the biological sample. With the filler liquid, the dilutions of the reagent liquid and the biological sample is maintained. Also, there is no loss of volume for the reagent liquid and the biological sample during aspiration and dispensation.

According to a fourth aspect, a sample container with a cap is provided, that in use contains a biological sample, the cap at least in part includes the cover layer as described in aspect one.

### BRIEF DESCRIPTION OF THE DRAWINGS

In the following drawings, same reference numbers generally refer to the same parts throughout. The drawings are not to scale, instead the emphasis is on describing the concept.

FIG. 1A is an elevation and cross-sectional view of the cartridge module under an embodiment of the invention where the cover layer is attached to the receptacle top openings.

FIG. 1B is an elevation and cross-sectional view of the cartridge module under an embodiment of the invention where the cover layer is attached to the cartridge module.

FIG. 1C is an elevation and cross-sectional view of the cartridge module under an embodiment of the invention where the cover layer is attached to the receptacles.

FIG. 2A and FIG. 2B show an elevation and cross-sectional front view and a collapsed side view respectively of an embodiment of the invention wherein the receptacle is made of a deformable material. FIG. 2C shows FIG. 2B in bloated condition.

FIG. 3A is a planar view of an embodiment of the cartridge module when without the cover layer. FIG. 3B is a perspective view of the cartridge module at FIG. 3A under the cover layer.

FIG. 4A shows the receptacles in a pile are made of a deformable material such that when with no contents, are in a collapsed condition. FIG. 4B illustrates an elevation and cross-sectional view of FIG. 4A where the receptacles are in bloated condition when containing the reagent liquid and the biological sample.

FIG. 5 is an elevation and cross-sectional view of an embodiment wherein a cap has overlaying regions of the cover layer.

FIG. 6A and FIG. 6B are elevation and cross-sectional views of embodiments where the cover layer respectively isolates individual and a plurality of receptacles in the cartridge module.

FIG. 7A and FIG. 7B illustrate elevation and cross-sectional partial views to describe the issues of using the hermetic cover layer.

FIG. 8A represents an elevational and cross-sectional view of an embodiment of a kit comprising the cartridge module, a syringe, and a pipette. FIG. 8B represents FIG. 8A in operations.

FIG. 9A is an elevational and cross-sectional view of a tube inside the receptacle and coated with the cover liquid in an embodiment. FIG. 9B represents FIG. 9A when the tube is retracted out of the receptacle and is coated with the cover liquid over the biological sample.

FIG. 10 represents an elevation and cross-sectional view of the cartridge module when with a scrubbing layer according to an embodiment of the invention.

FIG. 11A and FIG. 11B represent elevation and cross-sectional views of embodiments where the syringe has the cover liquid with one and two o-rings.

FIG. 12 illustrates an embodiment where the cover layer is tightly clamped by a rigid holding layer with rigid layer apertures for insertion of the tube.

FIG. 13 illustrates the embodiment if the cartridge module with the connecting air space further having a sealing layer over the receptacle top openings.

FIGS. 14A-14D illustrate an embodiment of operation of the apparatus, where FIG. 14A illustrates a state when the tube is inserted into the left outermost receptacle; FIG. 14B illustrates a state when the tube is lifted out of the left outermost receptacle, without the tube tip moving out of the connecting air space; FIG. 14C illustrates a state when the tube is again inserted into the right outermost receptacle; and FIG. 14D illustrates a state when the tube is lifted out of the right outermost receptacle, without the tube tip moving out of the connecting air space.

FIG. 15A and FIG. 15B represent elevation and cross-sectional views of embodiments before the amplification of the biological sample, where a liquid sealant is dispensed over or under the stack respectively.

FIG. 15C and FIG. 15D represent more embodiments of the receptacle for creating an air gap between the liquid sealant and the biological sample.

FIG. 16A illustrates a planar view of an embodiment of the cover layer where the pits holding the cover liquid are interconnected. FIG. 16B illustrates an elevation and cross-sectional view of the cover layer at the cutline A-B in FIG. 16A.

FIG. 17A and FIG. 17B are plan views of embodiments having through aperture or partially through aperture on the cover layer. FIG. 17C and FIG. 17D in elevation and cross-sectional views show the flexible nature of the cover layer at the aperture.

FIG. 18A, FIG. 18B, and FIG. 18 C illustrate elevation and cross-sectional views to describe the operation with an embodiment wherein the cover layer has a recessed top.

FIG. 19A to FIG. 19D are elevation and cross-sectional views according to an embodiment, for bending the tube as attached to the syringe. to accommodate in the cartridge before disposal.

FIG. 20A shows an embodiment where the tube ejects air into the hermetically isolated receptacle before aspirating as in FIG. 20B.

FIG. 21A in an elevation and cross-sectional view shows an embodiment where the tube dispenses into the hermetically isolated receptacle and FIG. 21B shows the tube sucking air from the receptacle after dispensing.

FIG. 22A in an elevation and cross-sectional view shows an embodiment where the tube dispenses into the hermetically isolated receptacle. FIG. 22B shows the tube retracted from the receptacle after dispensing. FIG. 22C shows the tube being briefly held within the cover liquid.

FIG. 23A to FIG. 23C are elevation and cross-sectional views according to an embodiment, for using a filter in the pipette.

FIG. 24A illustrates an embodiment of the tube head when attached to the tube. FIG. 24B illustrates an embodiment of the method of filling the liquid retaining space by the filler liquid.

FIG. 25 is a flow diagram for an embodiment of a method for nested PCR.

FIG. 26 illustrates a planar view of an embodiment of the cartridge module and a description of the sequences for extraction and sample loading for PCR.

FIG. 27 illustrates an elevational and cross-sectional view of the cover layer that allows a two-way passage of air but substantially restricts passage of aerosol, spilled reagent and the biological sample.

FIGS. 28A-28D are representations of FIGS. 15A-15D when without the connecting air space and with the porous cover layer on the receptacle top opening instead.

FIG. 29 shows an elevational cross-sectional figure for an embodiment with the scrubbing layer under the stack.

FIG. 30 is an elevation cross-sectional diagram of an embodiment, where the cartridge module further comprises at least one tube holder for holding the tube and being engageable with the syringe.

### DETAILED DESCRIPTION OF THE EMBODIMENTS

The following description presents several preferred embodiments of the present invention in sufficient detail such that those skilled in the art can make and use the invention.

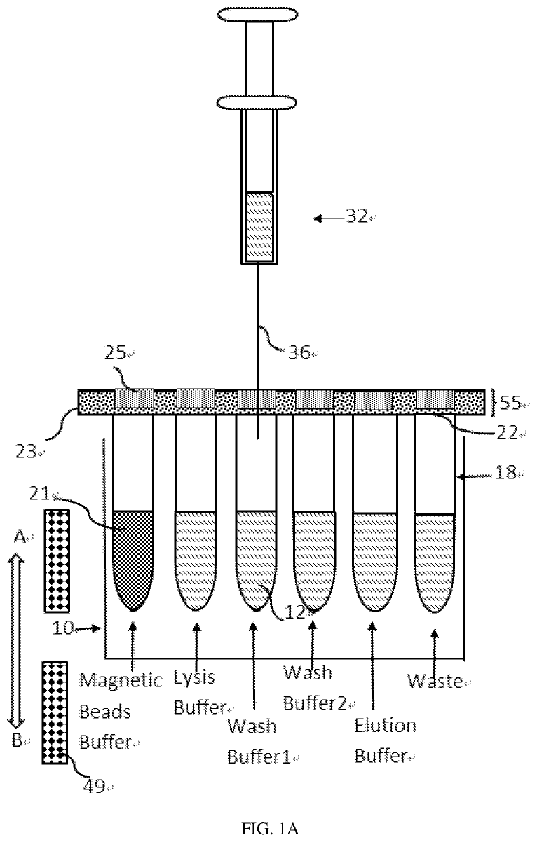

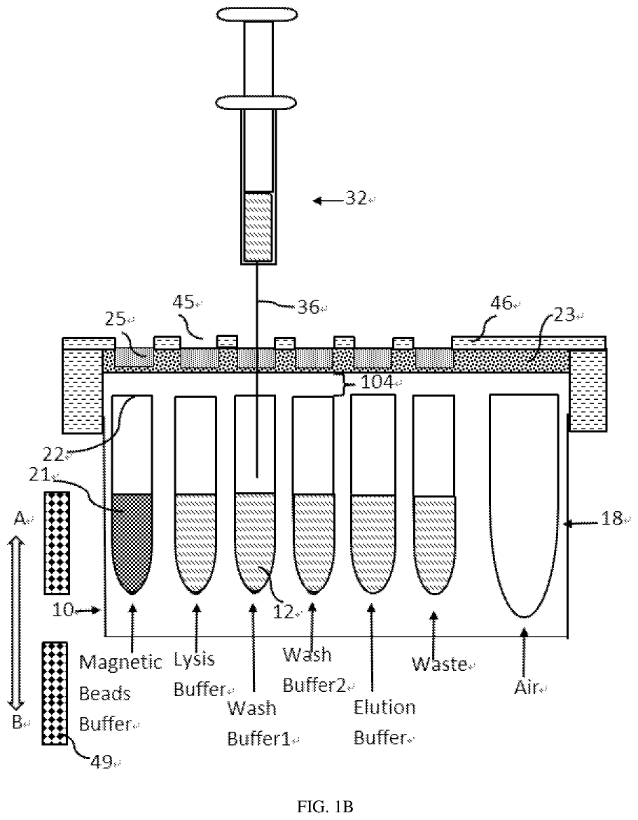

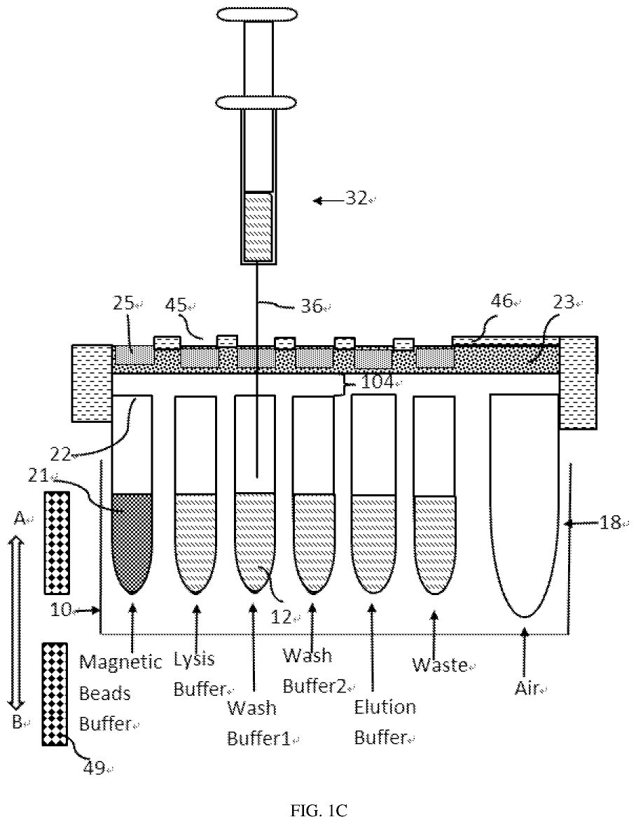

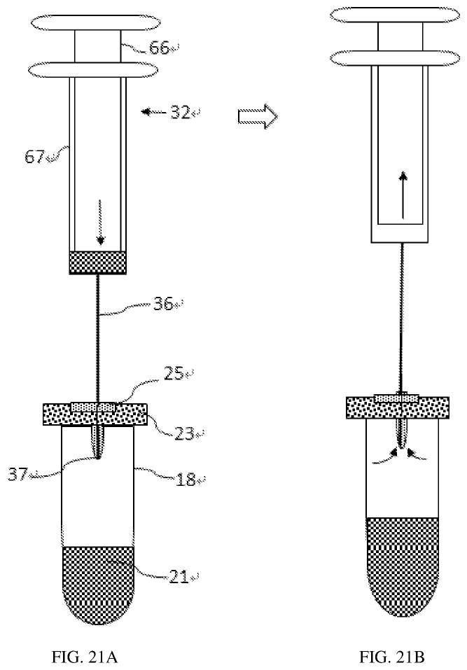

FIG. 1A illustrates an embodiment of the disposable cartridge module 10 for improving contamination control during nucleic acid analyses protocol, including nucleic acid extraction and/or nucleic acid amplification. The cover layer 23 is attached over the receptacle top openings 22. The receptacles 18 have no provision for liquid communication between the receptacles 18, while allowing for dispensing and aspirating liquid to and from each receptacle 18 only from a receptacle top opening 22. The tube 36 passes through the cover liquid 25 as held by the cover layer 23. The assay liquid 12 in the receptacles 18 are for nucleic acid extraction and/or nucleic acid amplification. The cover liquid 25 is provided on the cover layer 23 to form a stack 55 and is provided on the receptacles 18, the cover liquid 25 is immiscible and non-reacting with the reagent liquids 12 and the in use biological sample 21 containing nucleic acid. The cover liquid 25 is for a cover layer aperture 60 caused by piercing of the cover layer 23 by the tube 36 for dispensing and/or aspirating liquid into and from the receptacle 18. The tube 36, the cover liquid 25 and the cover layer 23 are mutually compatible to form the isolation to control the aerosol contamination into and from the receptacles 18. The cover liquid 23 is preferably immiscible with the reagent liquids 12 and the biological sample 21 and is wetting over the tube 36 and the cover layer 23 to fully cover the reagent liquid 12 or the sample 21 on the tube 36, when the tube 36 is taken out of the receptacle 18. The cover liquid 25 may be selected from any suitable material in the art including liquid silicone, oil and liquid wax. There are various kinds of reagent liquids 12 like lysis buffer, magnetic beads buffer, wash buffer, elution buffer, PCR reagent and the like. The function of the magnet 49 has been described under FIGS. 21A-21B. The cover layer 23 may comprise a sponge material at the stack 55 and over the receptacle top openings 22 for soaking the cover liquid 25, so that the cover liquid 25 is better secured within the cover layer 23. Some of the receptacles 18 may be loaded with reagent liquids 12 for the nuclear acid extraction and amplification. The cartridge module 10 may provide at least one empty slot 48 to accommodate at least one more of the receptacles 18 in use. The vertical and horizontal movements of the tube 36 need to be provided by an apparatus. The cover layer 23 may include an elastomeric material or a non-elastomeric material or a combination of both. The elastomeric material may be a non-porous material like natural or synthetic rubber or silicone or any other suitable material in the art. The elastomeric material may be a porous material like sponge. The non-elastomeric material may be a non-porous material like a plastic film or a metal film or their laminates or a porous material like a fiber mat. The cover layer 23 may be in the form of layers of more than one of the above materials. The tube 36 may be of metal or plastic or any other suitable material. The cartridge module 10 may include at least one receptacle 18 with no cover layer 23 for the user to isolate the receptacle 18 in use with the cover layer 23. The cover liquid 25 may be soaked in the cover layer 23 or sandwiched between the cover layer materials or form a stack 55 with the cover layer 23. Multilayers of the materials of the cover layer 23 is helpful particularly when the cover layer 23 includes non-elastomeric material. The cartridge module 10 may or may not include a syringe 32 or pipette 89 or reagent liquid 12. The tube 36, the syringe 32 or the pipette 89 may be placed in the cartridge module 10 for disposal. FIG. 1B illustrates an embodiment where the cover layer 23 is attached to the cartridge module 10. A connecting air space 104 is provided over the receptacles 18 and under the cover layer 23 to allow the plurality of receptacles 18 to be in air communication with one another. The empty receptacle 18 shown helps to add to the volume of the connecting air space 104. The cover layer 23 is tightly clamped by a rigid holding layer 46 with apertures 45 for insertion of the tube 36. The tube 36 passes through the cover liquid 25 as held by the cover layer 23. FIG. 1C illustrates an embodiment where the cover layer 23 is attached to the receptacles 18 with the connecting air space 104 over the receptacles 18 and under the cover layer 23 to allow the plurality of receptacles 18 to be in air communication with one another. A cartridge module 10 can house more than one syringe 32 or more than one tube 36 of different types or sizes as per the process requirements. A change mechanism for the syringe 32 and tube 36 is provided in the apparatus 100. The cartridge module 10 has a mechanism to house, release and receive: a) the syringe 32, b) the tube 36, and c) the syringe 32 mounted with the tube 36. The cartridge module 10 contains UV curable resin and a cleaning liquid for cleaning the tube 36. The cartridge module 10 contains magnetic beads for nucleic acid binding, or a porous filter like element for nucleic acid binding under vacuum, pressure and centrifugal force. The cartridge module 10 has a pattern to identify the position of the tube tip 37.

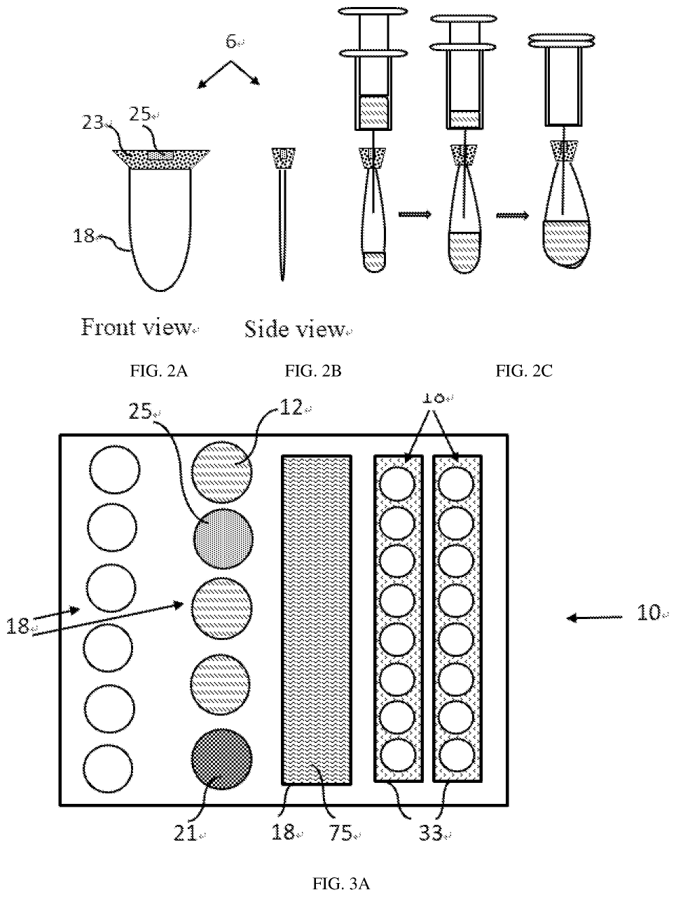

FIG. 2A represent elevation and cross-sectional view of a front view of the receptacle 18 according to an embodiment where the receptacle 18 is made up of a deformable material. FIG. 2B is a side view of the receptacle 18 of FIG. 2A when the receptacle 18 is in a collapsed condition. The receptacles 18 as indicated by the block arrows in FIG. 2C show how the deformable receptacle 18 from the collapsed condition 6 can bloat to increase the internal volume of the receptacle 18 to accommodate the reagent liquid 12 or the biological sample 21. The increase in the internal volume is achievable without being required to increase the air pressure inside the receptacle 18. The deformable material may be suitably selected from the state of the art and needs to be non-reactive with the reagent liquids 12 and the biological sample 21.

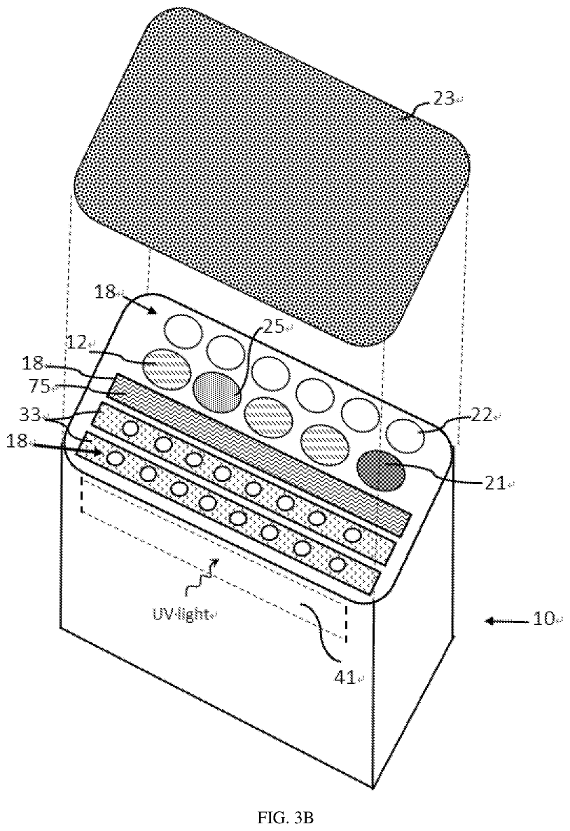

FIG. 3A illustrates an embodiment of the cartridge module 10 in a plan view. FIG. 3B is a perspective view of the cartridge module 10 with the cover layer 23 and a window 41 for receiving ultraviolet rays. The cartridge module 10 can be loaded and unloaded to/from an apparatus (not shown) for nucleic acid extraction and polymerase chain reaction PCR for amplification of nucleic acid. While some receptacles 18 are preloaded with reagent liquid 12 and the cover liquid 25, the rest are shown empty for the user to use for selected purposes of mixing the biological sample 21 with the reagent liquid 12. The biological sample 21 as defined here may or may not include one or more of the reagent liquids 12. The PCR reagent as the reagent liquid 12 is added to the biological sample 21 before amplification in an amplification module in the apparatus. The cover liquid 25 may be of more than one type as contained in different receptacles 18. There may be one or more receptacle holders 33 for holding one or more tubular receptacles 18. The receptacles 18 also may be in the form of or well plate with a plurality of tubular receptacles 18. The window 41 is shown to be transparent to ultraviolet rays in order to allow ultraviolet curing of a sealant liquid (not shown) when contained towards the top opening 22 of the receptacles 18. The source of ultraviolet rays may be adjustable to project the ultraviolet rays at an upward angle to protect the biological sample 21 contained at the bottom of the receptacle 18 to be unwantedly exposed to the ultraviolet rays provided in an apparatus. Specifically, the sealing and curing is to be conducted before the step of amplification. The receptacle holder 33 may be detachable from the cartridge module 10. The receptacle 18 may be fixedly attached to the receptacle holder 33. All the consumable components that are made to be in contacts with the biological sample 21 and the reagent liquid 12 are disposable after use. The cartridge module 10 includes a liquid bath medium 75 in at least one of the receptacles 18 for being heated during amplification of nucleic acid in the biological sample. An apparatus can then be pre-calibrated for heating this bath medium 75 and the user may only have to fine tune the calibrations onsite. The liquid bath medium 75 also allows easy transfer between the baths in an apparatus and the receptacle 18 in the cartridge module 18 via the receptacle top opening 22 and by the tube 36. During transport of the apparatus, the liquid bath medium 75 can be transferred back to the receptacle 18 in the cartridge module 10. Storing the liquid bath medium 75 in the receptacle 18 helps reduce evaporation effects as well when the apparatus is not in use.

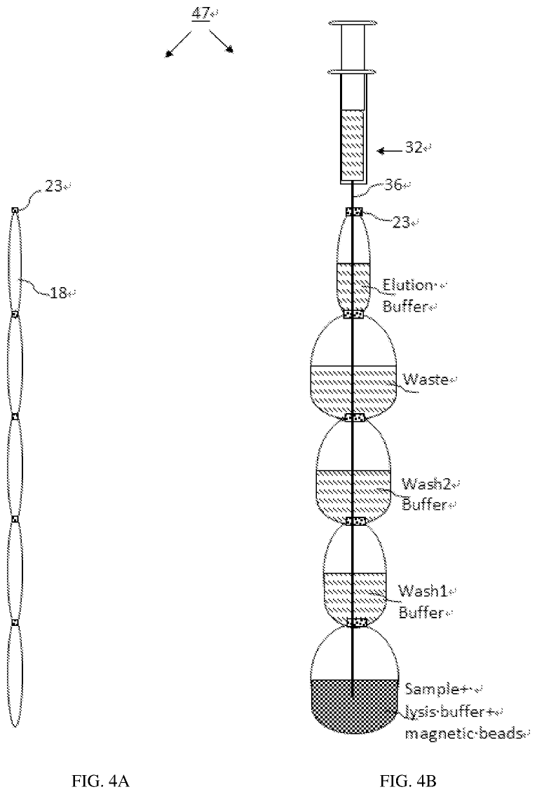

FIG. 4A shows the receptacles 18 in a pile are made of a deformable material such that when with no contents, are in a collapsed condition. FIG. 4B illustrates an elevation and cross-sectional view of FIG. 4A where the receptacles 18 are in bloated condition when containing the reagent liquid 12 and the biological sample 21.

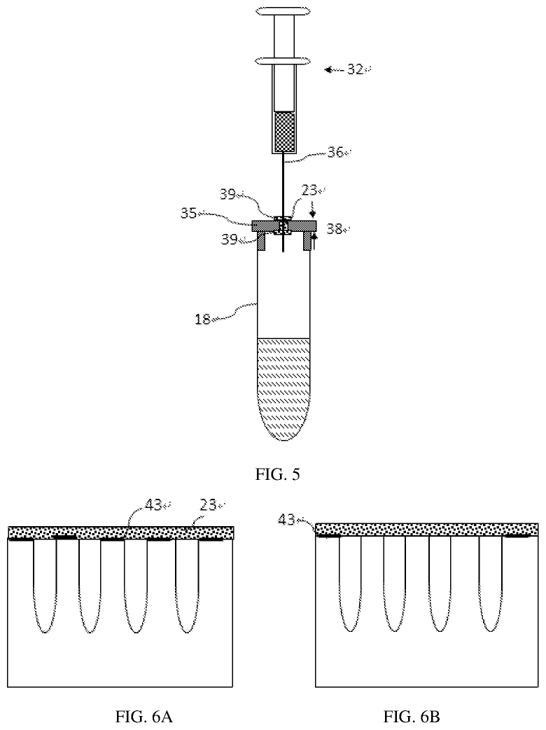

FIG. 5 illustrate an embodiment of the invention where a cap 35 for the receptacles 18 has the cover layer 23 embedded through the thickness 38 of the cap 35 with overlaying regions 39 over and under the cap 35. In portion the cap 35 has the cover layer 23 that can be penetrated by a tube 36, the cover layer 23 being embedded through the thickness 38 of the cap 23 and has a first and a second overlaying region 39 over and under the cap 35, the overlaying regions being to resist any relative movement between the cover layer 23 and the cap 35 during the piercing and the retraction by the tube 36.

FIG. 6A illustrates the cover layer 23 covering individual receptacles 18 in the cartridge module 10, by the attachment 43 to the cartridge module 10 around each receptacle top opening 22. FIG. 6B illustrates cover layer 23 covering a plurality of receptacles 18 in the cartridge module 10, by the attachment 43 to the cartridge module 10 around the plurality of the receptacles 18.

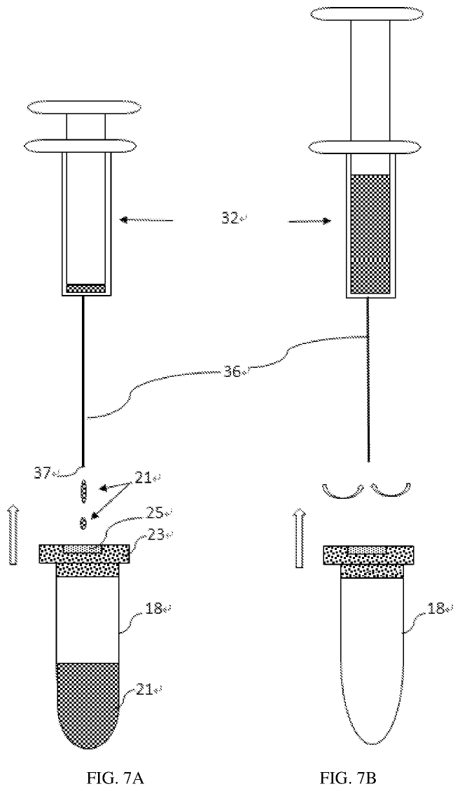

FIG. 7A and FIG. 7B illustrate certain practical issues with the hermetically isolating cover layer 23. As at FIG. 7A, the air pressure inside the receptacle 18 is increased due to dispensation by the tube 36. When the tube 36 is thereafter retracted out of the hermetically isolated cover layer 23, the biological sample 21 drips from the tube tip 37 at the atmospheric pressure, thereby exposing the biological sample 21 to the ambience and contaminating the ambience. Similarly, the air pressure inside the receptacle 18 is decreased due to aspiration by the tube 36. When the tube 36 is thereafter retracted out of the hermetically isolated cover layer 23, the ambient air forces into the tube 36 thereby causing chances for contamination into the tube 36 from the ambience.

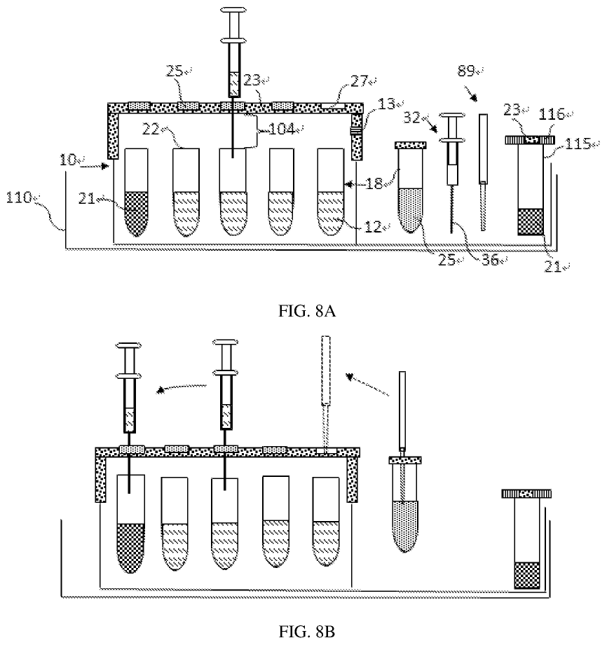

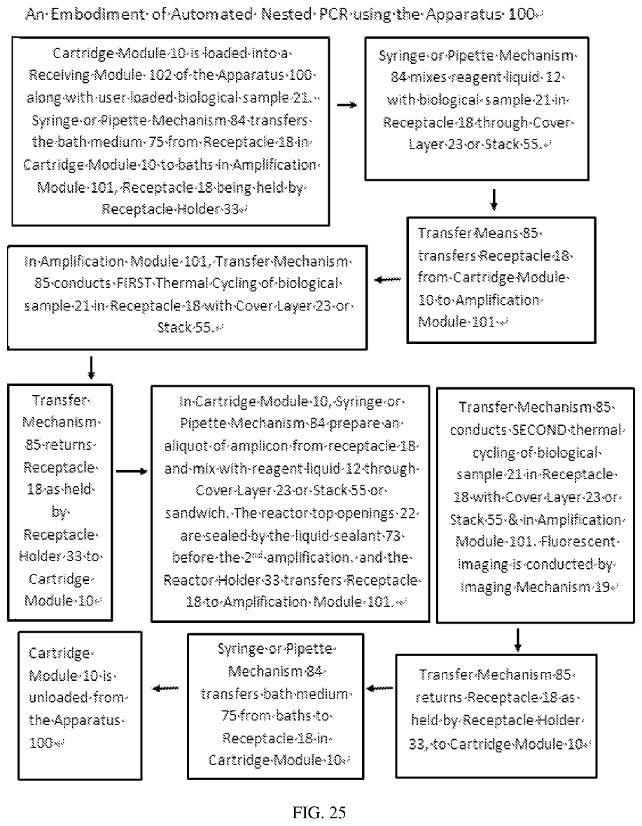

FIG. 8A illustrates an embodiment of a kit 110 comprising the cartridge module 10, a syringe 32 and a cover liquid pipette 89. The cover liquid 25 is provided in one of the receptacles 18 and the pipette 89 is used for dispensing the cover liquid 25 on the selected regions like pits 27 of the cover layer 23 while the receptacles 18 are in the cartridge module 10. FIG. 8B shows an embodiment where the pipette 89 dispenses the cover liquid 25 on the cover layer 23 and from the receptacle 18, as represented by the dashed arrow. The solid arrow represents a process where a reagent liquid 12 is aspirated from the receptacle 18 by the syringe 32 and dispensed into the receptacle 18 containing the biological sample 21. The pipette 89 may be in any form in the art. Dispensing the cover liquid 25 on the cover layer 23 inside the apparatus just before use is helpful in protecting the cover liquid 25 from getting displaced or wiped off during transport or handling. In this embodiment, the cartridge module 10 further accommodates a sample container 115 with a cap 116 that contains the biological sample 21 for analyses. The cap 116 includes the cover layer 23 for piercing the tube 36 to aspirate the biological sample 21 and dispense into the receptacle 18. When the cartridge module 10 is loaded into an apparatus 100 as described in FIG. 25, the syringe or pipette mechanism 84 therein is computer programmable to aspirate the biological sample 21 from the sample container 115 by the tube 36 and dispense into the receptacle 18. This improves the contamination control as the biological sample 21 is transferred into the receptacle 18 by the tube 36 while the sample container 115 is in the cartridge module 10. The cap 116 and the cover layer 23 in the cap 116 prevent any possible spillage of the biological sample 21 and aerosol leakage during the whole analysis until disposal of the cartridge module 10. This enables avoiding the complexities of manual transfer operation of the biological sample 21 into the receptacles 18 that requires a training to use a syringe 32 or a pipette 89 for dispensing into the receptacles 18 without contaminating the surroundings. This approach is particularly important for analyses of infectious biological samples 21.

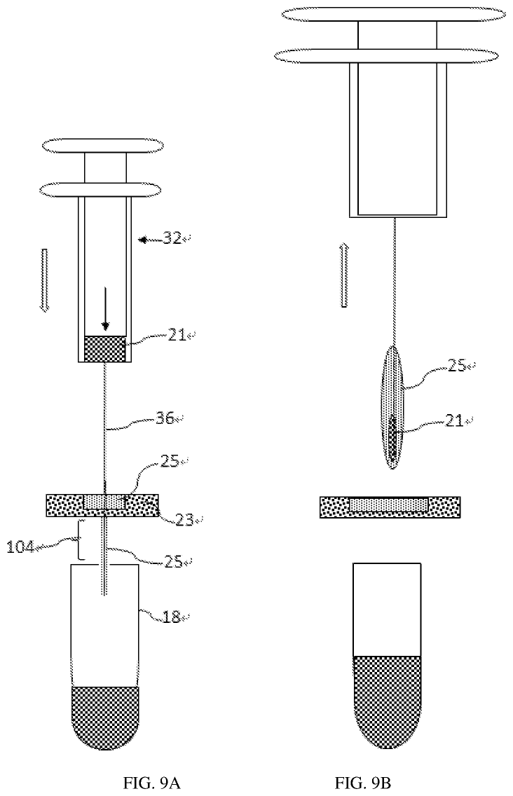

FIG. 9A illustrates the tube 36 of the syringe 32 inserted into the receptacle 15 through the stack 55 and dispensing the biological sample 21 into the receptacle 15. As the tube 36 passes through the cover liquid 25, an amount of the same covers the tube 36 that enters into the receptacle 18. As the cover liquid 25 is immiscible and non-reactive with the reagent liquids 12 and in use biological sample 21, there is no issue of contamination by the cover liquid 25. FIG. 9B illustrates the scenario when the tube 36 is retracted out of the receptacle 18 through the stack 55. As expected, by default the tube 36 is coated with a layer of the biological sample 21 and while the tube 36 passes through the cover liquid 25, a layer of the cover liquid 25 also covers the layer of the biological sample 21, as intended under this invention. Thus, the trace layer of the biological sample 21 is prevented from getting exposed to the ambience thereby reducing contamination. The cover liquid 25 also helps to seal the cover layer aperture 60 as created during the piercing of the tube 36. The cover liquid 25, the cover layer 23 and the tube 36 need to be mutually compatible in order to ensure the sealing of the cover layer aperture 60. The cover layer 23 is shown only in part.

FIG. 10 shows an embodiment where a scrubbing layer 28 is fixed over the stacks 55 such that the cover layer 23, the cover liquid 25 and the scrubbing layer 28 form a composite layer 30 for the tube 36 to pierce through to enter the receptacle 18. As shown, the scrubbing layer 28 helps to scrub off the excess cover liquid 25 sticking to the upper part of the tube 36 that could deplete the cover liquid 25 when in small quantity or when the tube 36 is long.

FIG. 11A illustrates an embodiment where the syringe 32 containing the biological sample 21 has two o-rings 68 between the plunger 66 and the barrel 67 of the syringe 32. The cover liquid 25 is provided in between the two o-rings 68 and away from a tube head 62 engaging to the barrel 67. FIG. 11B illustrates the scenario when the plunger 66 has been pushed further into the barrel 67 to dispense the biological sample 21. The cover liquid 25 within the o-rings 68 substantially covers the trace amounts of the biological sample 21 adhering to the inner wall of the barrel 67 and the outer wall of the plunger 66 hence the exposure of the biological sample 21 to the ambience remains substantially insignificant.

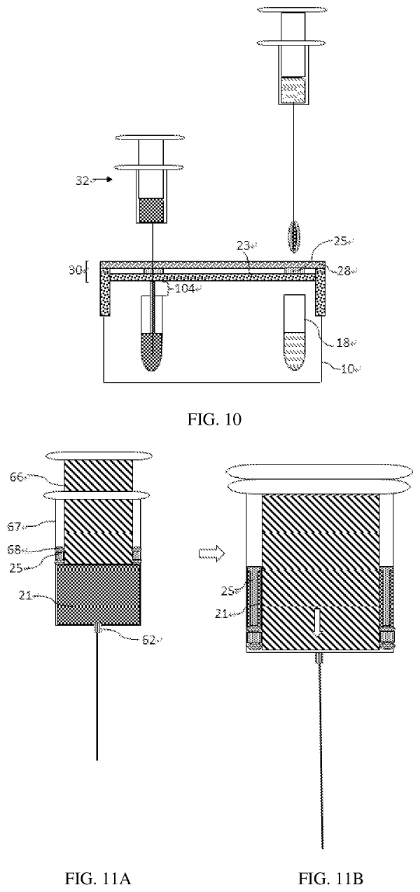

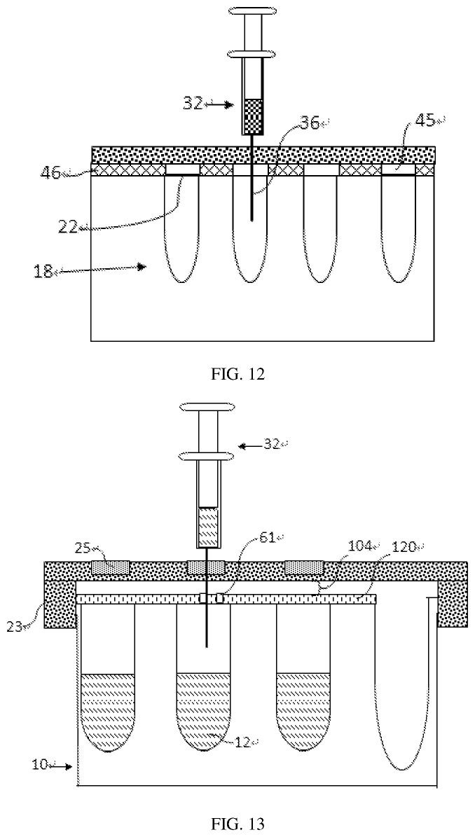

FIG. 12 illustrates an embodiment where the cover layer 23 is tightly clamped by a rigid holding layer 46 as positioned under the cover layer 23, the rigid holding layer 46 having rigid layer apertures 45 aligned over the receptacle top openings 22 for insertion of the tube 36 to penetrate the cover layer 23.

FIG. 13 illustrates the embodiment if the cartridge module 10 with the connecting air space 104 further having a sealing layer 120 over the receptacle top openings 22. The apparatus 100 is computer programmable so that the tube 36 pierces through the stack 55 and the sealing layer 120 to generate a vent aperture 61 in the sealing layer 120 over the receptacle top opening 22, so that the receptacle 18 is in air communication with the connecting air space 104 during aspiration or dispensation by the tube 36.

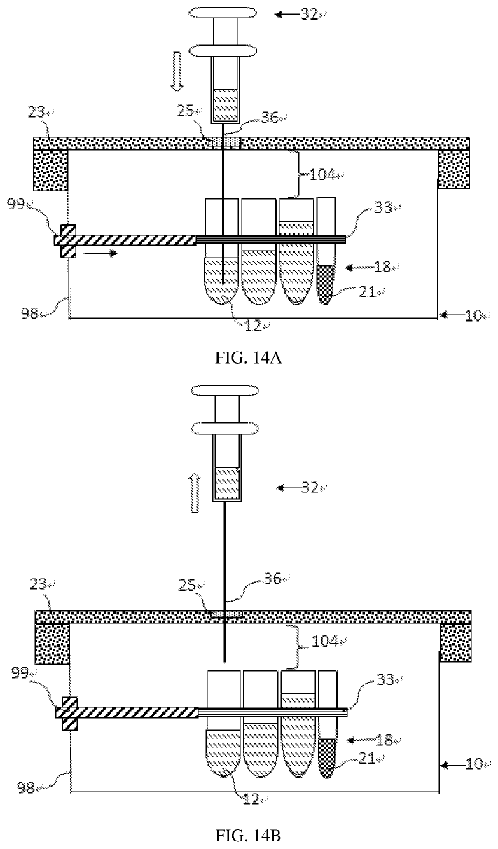

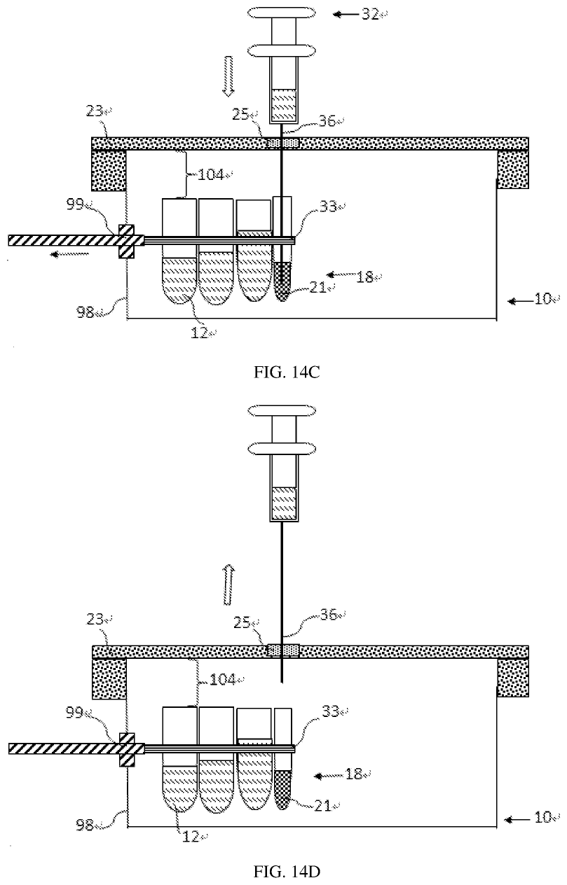

FIG. 14A to FIG. 14D illustrate an embodiment of operation of the apparatus 100. Herein, the cartridge module 10 is provided with a coupling mechanism 99 that is hermetically fitted on the cartridge module wall 98 and is connected to the receptacle holder 33 such that the coupling mechanism 99 is operable from outside the cartridge module 10 by the apparatus 100 to move the receptacle holder 33 horizontally to-and-fro and inside the cartridge module 10 to position the receptacle top openings 22 under the tube tip 37 while the receptacles 18 remain in the cartridge module 10. The syringe or pipette mechanism 84 is computer programmable to provide the vertical movements of the tube 36, such that during aspiration and dispensation of the reagent liquid 12 and the biological sample 21 from and into the receptacles 18, the tube 36 does not need to move out of the connecting air space 104. FIG. 14A illustrates a state when the tube 36 is inserted into the left outermost receptacle 18. FIG. 14B illustrates a state when the tube 36 is lifted out of the left outermost receptacle 18, without the tube tip 37 moving out of the connecting air space 104. FIG. 14C illustrates a state when the tube 36 is again inserted into the right outermost receptacle 18. FIG. 14D illustrates a state when the tube 36 is lifted out of the right outermost receptacle 18, without the tube tip 37 moving out of the connecting air space 104. According to an alternate embodiment (not shown), the vertical movements are provided to the cartridge module 10 instead of the tube 36. The hermetic coupling mechanism 99 helps the receptacles to be isolated from the ambience. Alternately the coupling mechanism 99 is operable from outside the cartridge module 10 to move the cartridge module horizontally such that the coupling mechanism 99 need not be hermetically attached to the cartridge module wall 98.

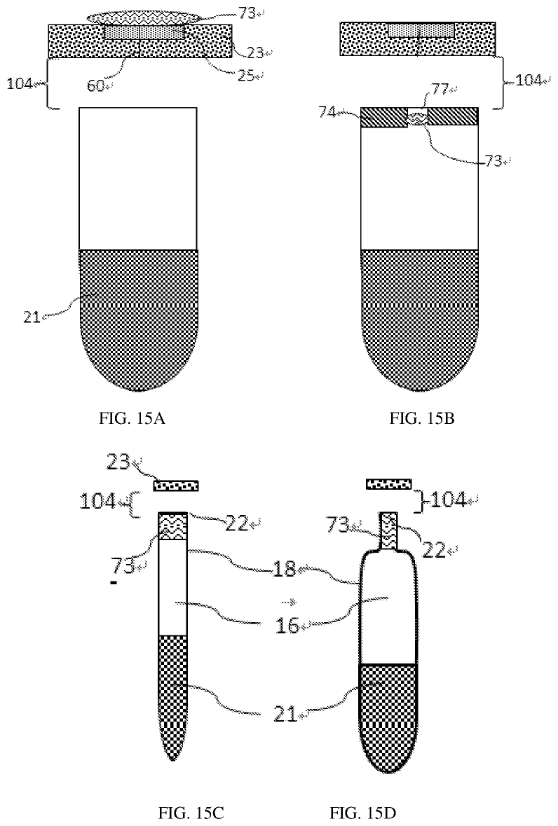

FIG. 15A illustrates disposal of a liquid sealant 73 over the stack 55 before amplification of the biological sample 21 inside the receptacle 18, followed by a cure of the liquid sealant 73 by a source of heat or light or chemical in the apparatus 100, while the receptacle 18 is in the cartridge module 10. FIG. 15B represents an alternate embodiment wherein the cover layer 23 has an aperture layer 74 underneath, the aperture layer 74 having a scaling aperture 75 that is scaled by injecting a liquid sealant 73 into the sealing aperture 75 before thermal treatment for amplification of the biological sample 21 in the receptacle 18. The liquid sealant 73 is injectable into the sealing aperture 75 by the tube 36 passed through the cover layer aperture 60. The liquid sealant 73 is cured by heat or light or chemical treatment while the receptacle 18 is in the cartridge module 10. This helps to prevent any aerosol and/or vapor generated during the thermal treatment from escaping out of the cover layer aperture 60. Any such contaminant entering the receptacles 18 from the ambience is also controlled. In case of nested PCR, the liquid sealant 73 is to be used before the second amplification only. If used before the first amplification and cured, adding the reagent liquids 12 through the hardened sealant liquid 73 before the second amplification will not be possible. In case of nested PCR, the sealant liquid 73 used in the first amplification can be in a liquid form or can be converted back to a liquid form (such as by heating the liquid sealant 73 in a wax form) after the first amplification is completed, to enable the tube 36 to aspirate and dispense from and to the receptacle 18 for PCR. If the liquid sealant 73 is cured before the first amplification, adding the reagent liquids 12 through the hardened liquid sealant 73 before the second amplification will not be possible. Optionally, without the use of the sealant liquid 73, the cover layer 23 may be directly pierced through by the tube 36 after the first amplification is completed, to enable the tube 36 to aspirate and/or dispense from/to the receptacle 18 before the second amplification. In an alternate embodiment, the sealant liquid 73 may be of high viscosity and may not be hardened or cured before the first amplification. In yet another embodiment, the sealant liquid 73 may be like a wax that can be hardened or cured before the first amplification and softened by heat after the first amplification to enable the tube 36 to aspirate and/or dispense from/to the receptacle 18 before the second amplification. The liquid sealant may be curable by ultraviolet rays or heat or a chemical and may be any suitable material in the art such as oil, polymer, monomer and wax. FIG. 15C and FIG. 15D illustrate a receptacle 18 where an air gap 16 is created and maintained between the liquid sealant 73 and the biological sample 21. Herein, a combination of the liquid sealant 73 and the receptacle 18 is selected such that the receptacle 18 can hold the liquid sealant 73 at the receptacle top opening 22 when dispensed therein. The syringe or pipette mechanism 84 is used to aspirate the liquid sealant 73 and dispense near the receptacle top opening 22. The biological sample 21 is dispensed into the receptacle 18 thereafter and through the liquid sealant 73. Herein, the cover liquid 25 is under the cover layer 23. The liquid sealant 73 may then be cured to harden before the amplification. The liquid sealant 73 may be in the form of wax or glue. The curing may be done by exposing the liquid sealant 73 to ultraviolet rays. The ultraviolet rays may be directed at the liquid sealant 73 at an upward angle so that the biological sample 21 does not get exposed as well. The inline curing helps to further integrate and automate the process before the thermal treatment to avoid the issue of vaporization from the biological sample 21 thereby causing contamination within an apparatus. Any other method in the art for the curing may be used as well, such as by chemical or heat treatment. In case of nested PCR, the liquid sealant 73 is to be used before the second amplification only. If used before the first amplification and cured, adding the reagent liquids 12 through the hardened sealant liquid 73 before the second amplification will not be possible. The liquid sealant 73 may be non-reacting and immiscible with the reagent liquids 12 and the biological sample 21. The air gap 16 serves to further encapsulate the biological sample 21 or the reagent liquid 12 from the ambience.

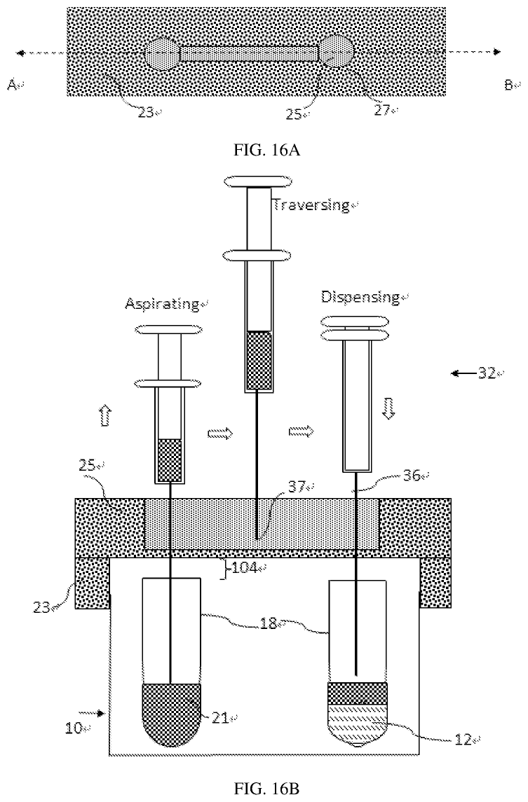

FIG. 16A illustrates a planar view of an embodiment of the cover layer 23 where the pits 27 holding the cover liquid 25 are interconnected. FIG. 16B illustrates an elevation and cross-sectional view of the cover layer 23 at the cutline A-B in FIG. 14A, where a tube tip 37 of the hollow tube 36 moves between the interconnected pits 27 while remaining dipped in the cover liquid 25 and without being exposed to the ambience. The tube 36 aspirates the biological sample 21 from the receptacle 18. As shown by the block arrows, after aspiration of the biological sample 21 by the tube 36, it is retracted out of the receptacle 18 and traversed over the receptacle 18. Thereafter, the tube 36 is made to enter the receptacle 18 by piercing the cover layer 23. The tube 36 then dispenses the biological sample 21 on the reagent liquid 12 in the receptacle 18. The tube 36 may aspirate and dispense multiple times to mix the two liquids. After dispensing, the tube 36 is meant to be retracted out of the receptacle 18 which is not shown here. Thus, during this operation, the tube tip 37 has remained either within the receptacles 18 or within the cover liquid 25. When out of one receptacle 18 and traversing to the other receptacle, the tube tip 37 is not exposed to the ambience. This prevents any unwanted drip of the biological sample 21 from the tube tip 37 to be exposed to the ambience.

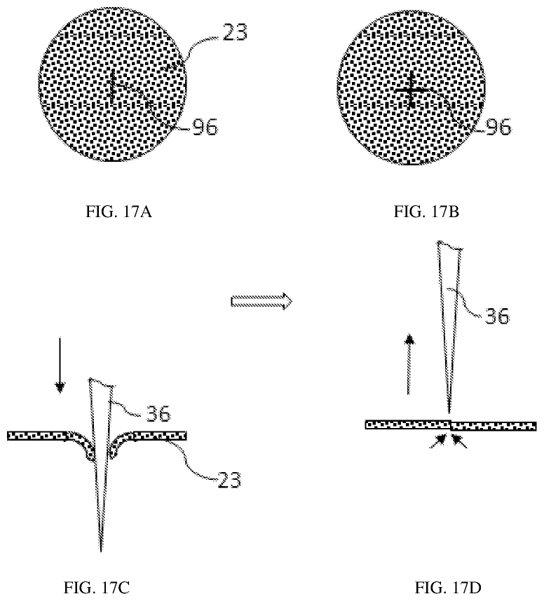

FIG. 17A in a plan view shows a through aperture 96 in unidirectional direction on the cover layer 23. FIG. 17B in a plan view shows a cross-shaped through aperture 96 on the cover layer 23, particularly for usage of blunt tubes 36 such as with pipettes 89. Any other shape may be used as well. FIG. 17C in an elevational and cross-sectional view shows the cover layer flexing downwards when the tube pierces through the aperture 96. FIG. 17D in an elevational and cross-sectional view shows the cover layer 23 flexing back to its substantially original planar shape when the tube 36 retracts out of the receptacle 18 through the aperture 96. Since the aperture 96 substantially flexes back to its original shape, the cross contamination between the interior of the receptacle 18 and the ambience is controlled. The cover liquid 25 may also be dispensed on the aperture 96 for enhanced isolation. The aperture 96 is required to be narrow enough to arrest any leakage of the cover liquid 25 into the receptacle 18.

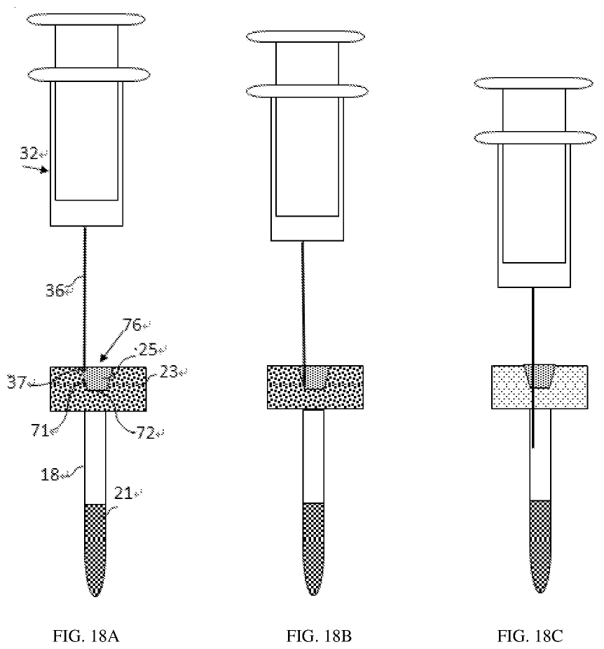

FIG. 18A, FIG. 18B, and FIG. 18C illustrate an embodiment of the receptacle 15 for nucleic acid analyses protocol including nucleic acid extraction or nucleic acid amplification. As shown, the receptacle 15 has a cover layer 23 covering the receptacle top opening 14 and isolating the receptacle 15. The cover layer 23 has a recessed top 76 for guiding an in-use tube tip 37 of a tube 36 before the tube tip 37 pierces the cover layer 23 through a base region 72 of the recessed top 76. The recessed top 76 has slanted sides 71 to provide a tapering towards the base region 72 serving as a guiding surface for the tube 36 to reach the base region 72, the base region 72 being same or smaller than the receptacle top opening 14. The slanted sides 71 are more rigid than the cover layer 23 so that as shown at FIG. 10, the tube tip 37 slides along the slanted sides 71 without piercing into the surface 71 and reaches the base region 72 for piercing through the cover layer 23 as shown at FIG. 18C. This provides better tolerance for misalignment of the tube tip 37 of the syringe 32 with reference to the small receptacle top opening 14. The slanted sides 71 may be made of materials such as metal and rigid plastic.

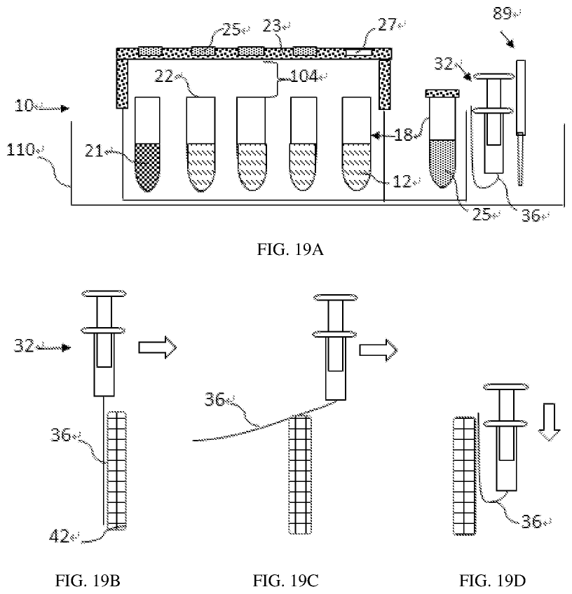

FIG. 19A shows an embodiment, where the tube 36 as attached to the syringe 32 is bent to accommodate within the cartridge module 10 before disposal of the cartridge module 10 after use. for bending the tube 36 as attached to the syringe. to accommodate in the cartridge module 10 before disposal. FIG. 19B, FIG. 19C and FIG. 19D illustrates with the block arrows how progressively the syringe or pipette mechanism 84 in the apparatus 100 is operated to press the tube 36 sideways and downwards against a fixed surface 42 in the cartridge module 10 or in the apparatus 100, to bend the tube 36. Thereafter the syringe 32 with the bent tube 36 is mounted in the cartridge module 10 as shown in FIG. 19A. The hollow tube 36 here is flexible and may be made of any suitable material such as metal.

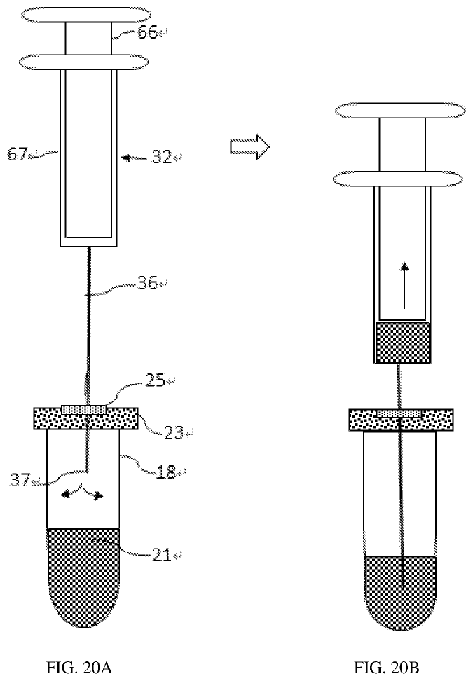

FIG. 20A illustrates that when the tube 36 is inserted into the receptacle 18 and before aspirating, the syringe operating mechanism 84 can be computer programmed such that when the tube tip 37 of the tube 36 has reached above the assay liquid 12 or the biological sample 21 in the receptacle 18, the syringe 32 conducts in an ejection mode for a predetermined time to create an excess air pressure in the hermetically isolated receptacle 18, the excess air pressure inside the receptacle 18 helps to at least partially release a vacuum created upon the following aspiration of the assay liquid 12 or the biological sample 21 by the tube 36 as in FIG. 20B.

FIG. 21A illustrates an embodiment of the apparatus. While the tube 36 is taken out of the receptacle 18 after dispensing, the syringe operating mechanism (not shown) can be computer programmed to operate according to the following step: when a tube tip 37 of the tube 36 has reached above the assay liquid 12 or the biological sample 21 in the receptacle 18, as illustrated in FIG. 21B, the syringe 32 conducts in a suction mode for a predetermined time to release at least a part of the excess air pressure in the hermetically isolated receptacle 18. The suction of the excess pressure is shown by the curved arrows. The excess air pressure is created due to dispensing the assay liquid 12 or the biological sample 21 in the hermetically isolated receptacle 18. Reducing the excess air pressure inside the receptacle 18 helps lesser assay liquid 12 or the biological sample 21 to be ejected out of the receptacle 18 when the tube tip 37 exits the cover layer 23.

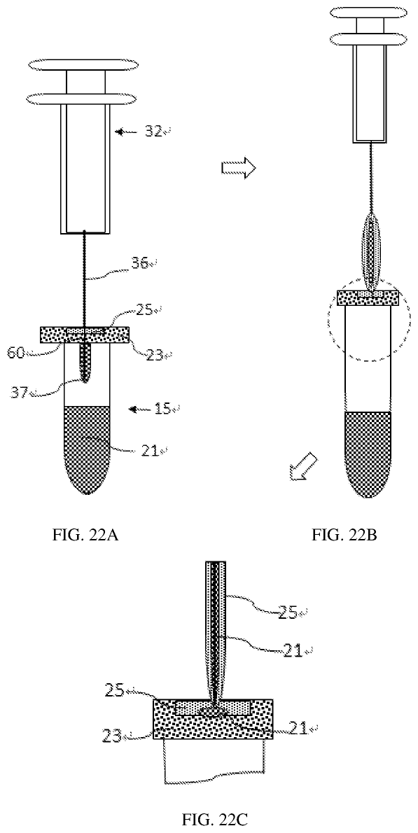

FIG. 22A illustrates an embodiment of an apparatus. While the tube 36 is taken out of the receptacle 18, the syringe operating mechanism can be computer programmed to operate according to the following step: when a tube tip 37 of the tube 36 has reached between the cover layer 23 and the cover liquid 25 as shown in FIG. 22B, the tube 36 holds for a predetermined time to release therein the assay liquid 12 or the biological sample 21 adhering to the tube 36. FIG. 22C is a blown-up view of the dashed circle region of FIG. 22B.

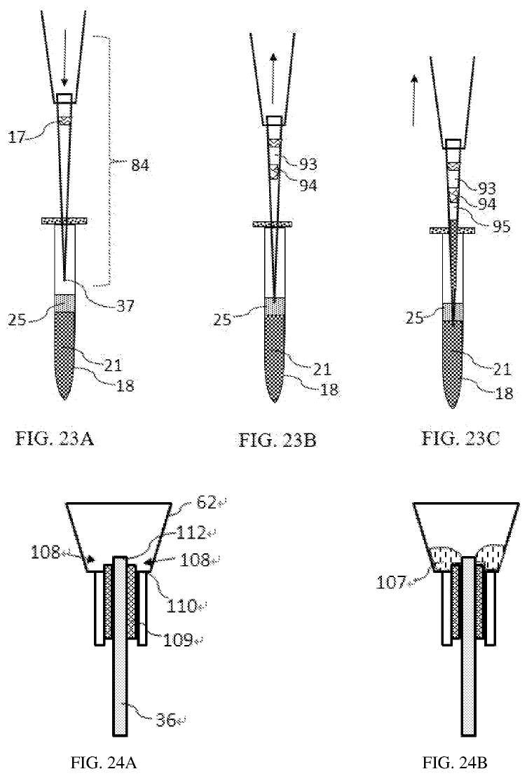

FIG. 23A illustrates a method wherein aspiration and dispensation of the biological sample 21 is done by the pipette mechanism 84 through a filter 17. FIG. 23B illustrates yet another embodiment where the aspiration and dispensation of the biological sample is done by the pipette mechanism 84, through a liquid layer 94 and the filter 17 maintaining a first gap 93 in between. FIG. 23C shows FIG. 23B with the biological sample 21 aspirated such that the biological sample 21, the liquid layer 94 and the filter 17 are all maintained separate from each other. The biological sample 21 maintains a second gap 95 from the liquid layer 94. This significantly reduces nucleic acid aerosol and/or vapor contamination during the aspiration and dispensation from and to the receptacle 18.

FIG. 24A describes an embodiment of the tube 36 as attached to the tube head 62 by a glue 109. The top tube tip 112 of the tube 36 protrudes above the bottom 110 of the tube head 62 to prevent the glue 109 from blocking the top tube tip 112. This forms a liquid retaining space 108. FIG. 24B illustrates the liquid retaining space 108 filled by a filler liquid 107 by aspirating the filler liquid 107 in the syringe 32 by the tube 36 and thereafter dispensing the filler liquid 107, such that the filler liquid 107 fills any existing liquid retaining space 108 in the tube head 62 that could otherwise retain the reagent liquid 12 and the biological sample 21 during the subsequent dispensation after aspiration. The filler liquid also replaces any reagent liquid 12 or the biological sample 21 from the previous processing steps that is retained in the liquid retaining space 108. This two-step cycle of aspiration and dispensation may be done a few times before one or more than one of the following steps: 1) aspirating purified biological sample 21 from elution buffer containing purified nucleic acids. In a direct PCR or other direct amplification protocols, the nucleic acid purification steps are not required, 2) aspirating PCR reagent liquid 12 to prepare a final reaction mix containing purified nucleic acids, 3) aspirating a final reaction mix to load into a PCR receptacle 18, and 4) any step in a nested PCR protocol. Nucleic acid extraction steps or PCR steps are optional. Other sample processing can be carried out on the cartridge module 10 with the tube 36, including a direct PCR which releases nucleic acids from a sample without purification steps, isothermal PCR with and without nucleic acid purification steps.

FIG. 25 is a flow diagram in an apparatus 100 for nested PCR according to an embodiment of the invention. The apparatus 100 is for improving contamination control during nucleic acid analyses protocol including nucleic acid extraction, nucleic acid amplification and detection. In this embodiment, the cartridge module 10 is loaded into the apparatus 100 via a receiving module 102 with the biological sample 21. The syringe or pipette mechanism 84 transfers the bath medium 75 from the receptacle 18 in the cartridge module 10 to the baths (not shown) in an amplification module 101. The syringe or pipette mechanism 84 is for operating the hollow tube 36 along with the syringe 36 or the pipette 89 to aspirate and dispense through the cover layer 23 from the receptacle top opening 22 at predefined locations. The syringe or pipette mechanism 84 mixes biological sample 21 with reagent liquid 12 in receptacle 18 with cover layer 23, for extraction and PCR. The transfer means 85 then transfers the receptacle 18 from the cartridge module 10 to the amplification module 101. In the amplification module 101, the transfer mechanism 85 conducts a first thermal cycling of the biological sample 21 in the receptacle 18 with the cover layer 23 and as held by the receptacle holder 33. Alternately, the amplification module 101 may provide isothermal heating instead of cyclic heating as shown, for amplification of the nucleic acid in the receptacle 18. The transfer mechanism 85 then returns the receptacle 18 as held by the receptacle holder 33 to the cartridge module 10. The syringe or pipette mechanism 84 further mixes the reagent liquid 12 to the biological sample 21 through the cover layer 23, with the receptacle 18 in the cartridge module 10. The transfer mechanism 85 then conducts a second thermal cycling of the biological sample 21 in the receptacle with the cover layer 23 and in the amplification module 101. A fluorescent imaging is conducted by an imaging mechanism 19 during or after the amplification. The robotically controlled transfer mechanism 85 is configurable to position the receptacle 18 as held by the receptacle holder 33 outside the cartridge module 10, as in this embodiment the amplification is conducted outside the cartridge module 10. Alternately, the receptacle 18 may remain in the cartridge module 10 to undergo amplification by the apparatus 100. The transfer mechanism 85 thereafter returns the receptacle 18 as held by the receptacle holder 33, to the cartridge module 10. The syringe or pipette mechanism 84 then transfers the bath medium 75 from the baths to the receptacle 18 in the cartridge module 10. Thereafter the cartridge module 10 is unloaded from the apparatus 100. The cartridge module 10 is disposed once the reagent liquid 12 is consumed. The apparatus 100 is computer programmable to treat the biological sample 21 with the reagent liquid 12 while the receptacles 18 are in the cartridge module 10, and conduct at least one of nucleic acid extraction, nucleic acid amplification and detection for the biological sample 21. The embodiment for this nested PCR has wide application for fast point of care testing (POCT). The optical imaging may be conducted by the imaging mechanism 19 with the receptacle 18 inside or outside the cartridge module 10. The concept of using the tube 36 along with the cover layer 23 and the cover layer 23 with the cover liquid 25 as in the preceding description enables extraction and nested PCR with dual amplification to be conducted within the apparatus 100 without having to take the cartridge module 10 out in between. In a nested PCR, receptacles 18 for the first amplification, mixing with the reagent 12 for the second PCR, and the second amplification shall be all covered by one or more cover layers 23 either on individual receptacles 18 or over all the receptacles 18.

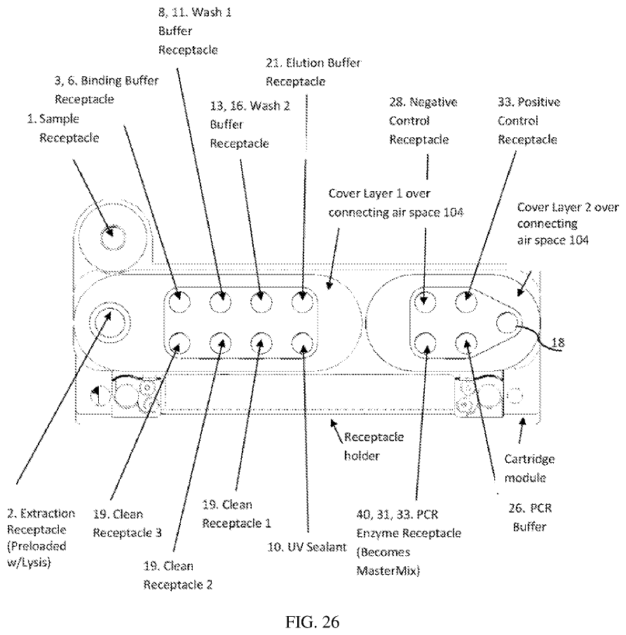

FIG. 26 in a planar view of the cartridge module, in part, describes a flow diagram for an extraction sequence and PCR. FIGS. 1A-1C also may be referred to.

### P26 Extraction Sequence

1. Mix biological sample receptacle 18 using tube 36
2. Aspirate biological sample 21 into lysis receptacle 18, and mix using tube 36
3. Aspirate binding buffer (magnetic beads) into lysis receptacle 18 and mix using tube 36
4. Move magnet 49 up from position B to position A, and hold at position A for a predetermined time
5. Mix solution in lysis receptacle 18 while the magnet 49 is up
6. Discard supernatant into waste receptacle 18
7. Move magnet 49 down from position A to position B
8. Aspirate wash 1 buffer and dispense into lysis receptacle 18, and mix using tube 36
9. Move magnet 49 up from position B to position A, and hold at position A for a predetermined time
10. Mix solution in lysis receptacle 18 while magnet 49 is up
11. Discard supernatant into waste receptacle 18
12. Move magnet 49 down from position A to position B
13. Aspirate wash 2 buffer and dispense into lysis receptacle 18, and mix using tube 36
14. Move magnet 49 up from position B to position A, and hold at position A for a predetermined time
15. Mix solution in lysis receptacle 18 while magnet 49 is up
16. Discard supernatant into waste receptacle 18
17. Move magnet 49 down from position A to position B
18. Start heating lysis receptacle to a predetermined temperature
19. Wash tube 36 at Clean 1 receptacle 18, Clean 2 receptacle 18, and Clean 3 receptacle 18
20. Stop heating lysis receptacle 18
21. Aspirate 100 ul elution buffer into lysis receptacle 18 and mix using tube 36
22. Move magnet 49 up from position B to position A, and hold at position A for a predetermined time
23. Mix solution in lysis receptacle 18 while magnet 49 is up
24. Move magnet 49 down from position A to position B
25. Eluent is extracted out for PCR
   Herein, all steps of mixing are by a few rounds of aspiration and dispensation by the tube 36. Herein, under step 1, the biological sample 21 is mixed within the receptacle 18. Under step 2, a volume of the sample 21 is aspirated and dispensed into the Extraction receptacle 18 containing Lysis and mixed. Under step 3, another volume of a binding buffer with magnetic beads is added into the lysis receptacle 18 by the tube 36. Under step 4, a magnet 49 is then moved up from a position B to a position A adjacent to the lysis receptacle 18 and held there for a predetermined time. Under step 5, the solution in lysis receptacle 18 is mixed while the magnet 49 is at position A, when the magnetic beads with the DNA and RNA attached are seated on the walls of the Lysis Receptacle 18 and towards the magnet 49. Under step 6, the supernatant/waste liquid is then transferred by the tube 36 from the Lysis Receptacle into Binding Buffer receptacle 18. Under step 7, the magnet 49 is then moved down from position A to position B. Under step 8, a volume of wash 1 buffer is aspirated from the wash 1 buffer receptacle 18 and dispensed into lysis receptacle 18, followed by mixing. Under step 9, the magnet 49 is again moved up from position B to position A and held at position A for a predetermined time. Under step 10, the solution in the lysis receptacle 18 is mixed while the magnet 49 is up. Under step 11, the supernatant/waste liquid is then transferred by the tube 36 from the Lysis Receptacle 18 into Wash 1 Buffer receptacle 18. Under step 12, the magnet 49 is then moved down from position A to position B. Under step 13, a volume of wash 2 buffer is aspirated from the wash 1 buffer receptacle 18 and dispensed into lysis receptacle 18, followed by mixing. Under step 14, the magnet 49 is again moved up from position B to position A and held at position A for a predetermined time. Under step 15, the solution in the lysis receptacle 18 is mixed while the magnet 49 is up. Under step 16, the supernatant/waste liquid is then transferred by the tube 36 from the Lysis Receptacle 18 into Wash 2 Buffer receptacle 18. Under step 17, the magnet 49 is then moved down from position A to position B. Under step 18, the lysis receptacle 18 is then heated to a predetermined temperature for a predetermined time, until washing in Clean 3 receptacle is completed. Under step 19, the tube 36 is washed by a few rounds of aspiration and dispensation in Clean 1 receptacle 18, followed by Clean 2 receptacle 18, and followed by Clean 3 receptacle 18. Under step 20, the heating of the lysis receptacle 18 is then stopped. Under step 21, a volume of elution buffer from the Elution Buffer Receptacle 18 is aspirated by the tube 36 into the lysis receptacle 18 and mixed. Under step 22, the magnet 49 is again moved up from position B to position A and held at position A for a predetermined time. Under step 23, the solution in the lysis receptacle 18 is mixed while the magnet 49 is up. Under step 24, the magnet 49 is then moved down from position A to position B. Under step 25, the Eluent is then aspirated out by the tube 36 for PCR. FIGS. 24A-24B also illustrate the sample loading sequence for PCR, after the preceding extraction sequence. Herein under step 26, the PCR Buffer is mixed in the PCR Buffer receptacle 18. Under step 40, a predetermined volume of the PCR Buffer is aspirated from the PCR Buffer Receptacle 18 into the PCR Enzyme Receptacle 18 containing PCR Buffer Enzyme and mixed to form a MasterMix Solution. Under step 28, a predetermined volume of the MasterMix solution from the PCR Enzyme Receptacle is aspirated and dispensed into a Negative Control Receptacle 18 and mixed. Under step 29, a predetermined volume of the mixed Negative Control Mastermix is aspirated and dispensed into a Glass Capillary Receptacle 1 (not shown). Under step 30, the magnet 49 is again moved up from position B to position A and held at position A for a predetermined time. Under step 31, a predetermined volume is aspirated from the Extraction Tube and dispensed into a PCR Enzyme Receptacle and mixed. Under step 32, a predetermined volume of the biological sample 21 from the PCR Enzyme Receptacle 18 is aspirated and dispensed into a Glass Capillary Tube 2 (not shown). Under step 33, a predetermined volume from PC Buffer Receptacle 18 is aspirated and dispensed into PCR Enzyme Receptacle 18 and mixed. Under step 33, a predetermined amount of the solution is aspirated from PCR Enzyme receptacle and dispensed into Glass Capillary Receptacle 3, which serves as a positive control—PC. Under step 34, a predetermined amount is aspirated from UV Buffer receptacle 18 and dispensed into the Glass Capillary receptacle 18. Under step 35, the Glass Capillary receptacles 18 are thereafter sealed using UV light. Then the PCR is conducted.

There may be a peeling layer (not shown) over the stack 55 to prevent the cover liquid 25 from displacement when the receptacle 18 is not in upright position, the peeling layer 31 being peelable before the piercing. Alternately the peeling layer is pierceable by suitable tube 36 for dispensing and/or aspirating liquids to and from the receptacle 18 through the stack 32.

The structure and configuration of the baths disclosed under this invention do not limit the scope of achieving any kind of thermal profile. Any user specified thermal profile may be attained by suitably placing the receptacles 18 in a specified sequence and for specified time periods in the baths that are maintained at pre-determined temperatures. The receptacle 18 can rapidly cross-over the extra baths (if any) along the line of the baths that do not contribute to the thermal profile.

The tube tip 37 may be wedge-shaped for easier piercing of the cover layer 23. Typically, the outer diameter of a tube 36 or a tube 36 with beveled or wedge-shaped tip is between 0.1 mm and 5 mm, and is preferred to be less than 1 mm. The thicker the cover layer 23, the higher is the chance that the cover layer aperture 60 can be fully closed upon removal of the tube 36. The preferred thickness of the cover layer 23 is larger than 1 mm. The cover liquid 25 remains in liquid form for at least one month, when stored at 10 degrees Celsius to 50 degrees Celsius.

A vibration mechanism (not shown) may be used for vibrating the receptacles 18 at predetermined frequency, amplitude and time for dispersing in use magnetic beads within the biological sample 21. This is an alternate method to mixing by aspirating and dispensing. A press-release mechanism (not shown) for alternately pressing and releasing the receptacles 18 in the cartridge module 10 may also be used instead, for mixing the biological sample 21 in the receptacle 18 that are flexible or elastically deformable under the pressing. The apparatus 100 may further comprise a motorised stage (not shown) to position the receptacle top openings 22 under the tube tip 37, while the receptacle 18 remains in the cartridge module 10.

The receptacles 18 may be of any shape and be made of any material in the art. The receptacle 18 may be made in the form of a glass capillary of small diameters such as 0.1 mm-4 mm OD and 0.02 mm-3 mm ID. Simultaneous PCR can be advantageously conducted for non-identical biological samples 21 if the bath temperatures are suitable.

From the foregoing description, it will be understood by those skilled in the art that many variations or modifications in details of design, construction and operation may be made without departing from the present invention as defined in the claims.

The terminology ‘substantially’ in the claims is to be interpreted as: intended to be completely or 100%, with small deviations or offsets for practical reasons.

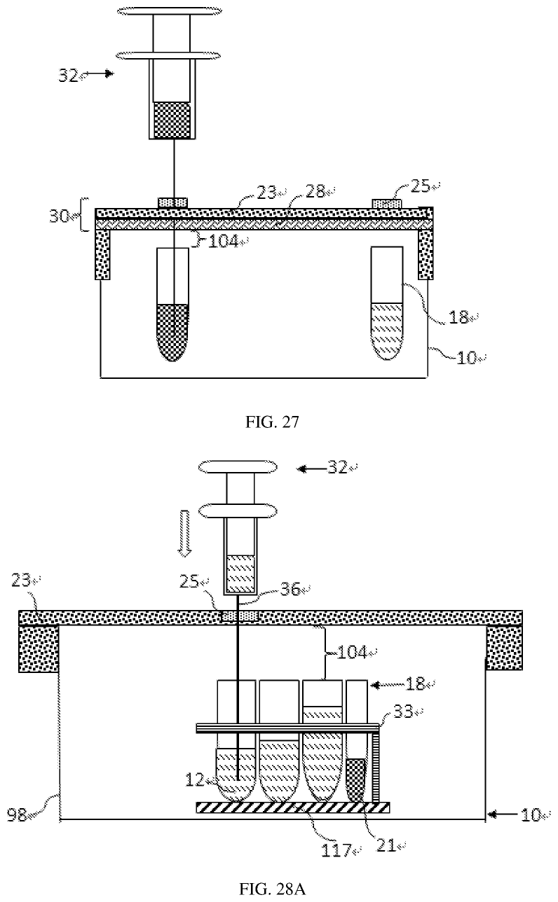

FIG. 27 shows an elevational cross-sectional figure for an embodiment with the scrubbing layer 28 under the stack 55 and over the connecting air space 104. The scrubbing layer 28 along with the cover layer 23 substantially scrubs off the excess cover liquid 25 sticking to the outer part of the tube 36 that could otherwise undesirably increase the volume of the liquid within the receptacle 18. Both the embodiments shown in FIG. 10 and FIG. 27 may be provided together to exploit the specified advantages. The scrubbing layer 28 over or under the stack 55 may be with or without a gap from the stack 55.

It may be appreciated that when the apparatus 100 is computer programmable to position the cartridge module 10 such that during aspiration and dispensation of the reagent liquid 12 and the biological sample 21 by the syringe or pipette operating mechanism 84 from and into the plurality of receptacles 18, the portion of the tube 36 that contacted the biological sample 21 in the receptacle 18 does not move out of the connecting air space 104, the syringe or pipette operating mechanism 84 also needs to move together with the cartridge module 10.

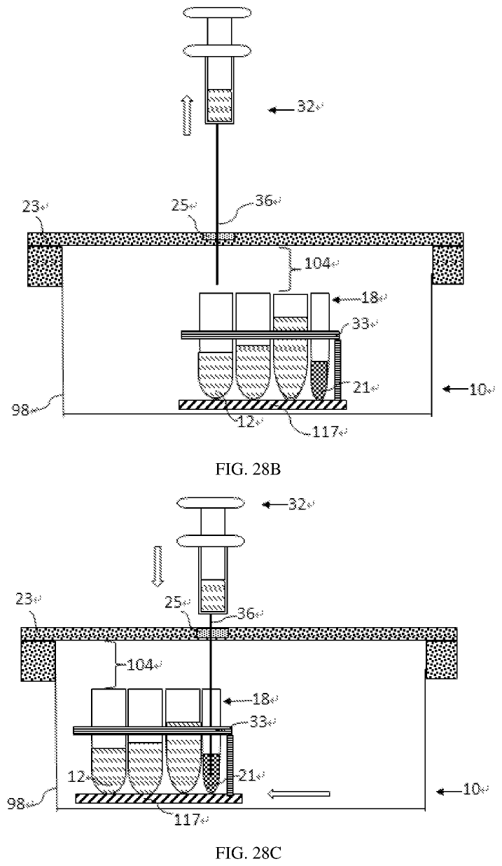

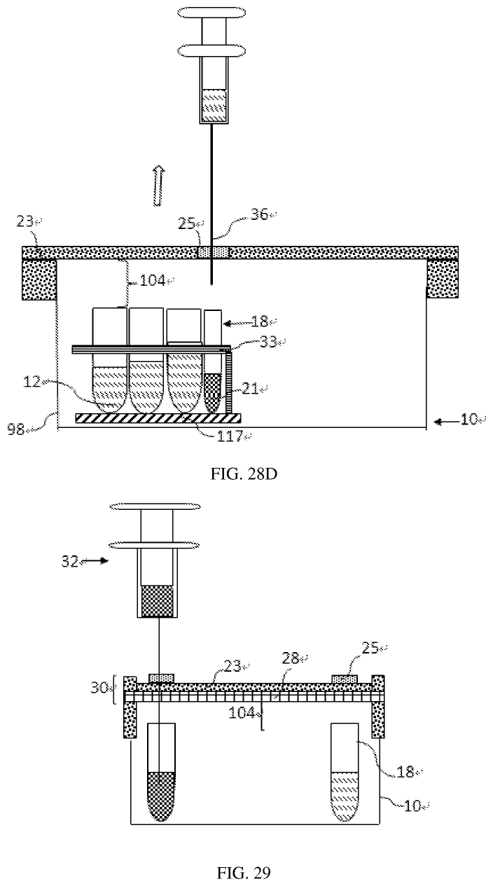

FIG. 28A to FIG. 28D illustrate a preferred embodiment of operation of the apparatus 100. Herein, the cartridge module 10 is provided with a translatory mechanism 117 within the cartridge module 10. The translatory mechanism 117 is connected to the receptacle holder 33 and is operable within the cartridge module 10, by the apparatus 100, to move the receptacle holder 33 horizontally to-and-fro and inside the cartridge module 10 to position the receptacle top openings 22 under the tube tip 37 while the receptacles 18 remain in the cartridge module 10. The syringe or pipette mechanism 84 is computer programmable to provide the vertical movements of the tube 36, such that during aspiration and dispensation of the reagent liquid 12 and the biological sample 21 from and into the receptacles 18, the tube 36 does not need to move out of the connecting air space 104. FIG. 28A illustrates a state when the tube 36 is inserted into the left outermost receptacle 18. FIG. 28B illustrates a state when the tube 36 is lifted out of the left outermost receptacle 18, without the tube tip 37 moving out of the connecting air space 104. FIG. 28C illustrates a state when the tube 36 is again inserted into the right outermost receptacle 18. FIG. 28D illustrates a state when the tube 36 is lifted out of the right outermost receptacle 18, without the tube tip 37 moving out of the connecting air space 104. Thus a substantially horizontal movement of the receptacle holder 33 is provided within the cartridge module 10 such that during the movement all physical elements within the cartridge module 10 remain within the cartridge module 10. This ensures that during the movement, the interior of the cartridge module 10 to remain isolated from the ambience, thereby arresting cross contamination between the ambience and the interior of the cartridge module 10. The vertical movements may be provided to the cartridge module 10 instead of the tube 36.

The cover layer 23 when elastomeric at least where the tube 36 pierces the cover layer 23 and retracts helps in the blockage of aerosol exchange and the spillage of the reagent liquid 12 or the biological sample 21 from the receptacles 18 or the cartridge module 10 into the ambience. Elastomers are rubber-like solids with elastic properties. Aerosols are typically defined as solid/aqueous particles suspended in the air and are typically of size 1⁻⁹m-10⁻⁴m, as generated by natural and anthropogenic sources. A typical diameter of air molecule is reported to be about 4×10⁻¹⁰ m.

The syringe pick-up module (not shown) in the apparatus 100 picks up at least one syringe 32 or at least one pipette 89 from the cartridge module 10 and releases back into the cartridge module 10 after one or more operations such as the aspiration and the dispensation. The tube pick-up module (not shown) in the apparatus 100 is for mounting the tube 36 as provided in the cartridge module 10 onto the syringe 32 or the pipette 89.

The transfer mechanism 85 picks up the reactor holder 33 for thermal cycling and returns to the cartridge module 10 after the cycling. The syringe pick-up module and the tube pick up module pick up the syringe 32 and the tube 36 by the syringe 32 from the cartridge module 10 and put back both into the cartridge module 10 after the test, after bending the tube 36. When returning the tube 36 to the cartridge module 10, the tube 36 as attached to the syringe 32 or the pipette 89 may be dipped in a protective liquid (not shown) to be covered with the protective liquid to cover the tube tip 37 to prevent contamination during the tube 36 or the syringe 32 returning process. The protective liquid may be a viscous UV glue.

The cover layer 23 may comprise of multiple layers and be in multiple phases such as the cover liquid 25, an air gap, a pierceable metal foil such as for sealing the receptacle 18, sponge and a hermetic material like rubber or plastic or silicone. The cover layer 23 may include paper, plastic or aluminum. The cover layer 23 may be a special paper that satisfies the qualities as defined under the invention. The cover liquid 25 may be a gel, particularly when soaked in the cover layer 23 or sandwiched within the cover layer 23. The tube 36 preferably has a sharp tip 37 for piercing the cover layer 23 with minimum damage in the cover layer 23. The tube 36 may be a hypodermic needle. The tube 36 may preferably be disposed after use. If the tube 36 is reused, any trace of DNA left would be amplified and cause contamination. In order to assist the pressure regulation inside the receptacle 18, the cover liquid 25 does not fully block the cover layer 23 over receptacle top opening 22, so that air can still pass through the porous layer. Apart from closing the cover layer aperture 60, the cover liquid 25 is also capable of closing the cracks generated if any in the cover layer 23 during the piercing of the tube 36. The sponge in the cover layer 23 may be soaked in cover liquid 25. The cover layer 23 may include paper, plastic or aluminum. The cover layer 23 may be a special paper that satisfies the qualities as defined under the invention. The PCR tube can be sealed by a UV curable resin, oil, gel, polymer or monomer liquid, etc. The cover liquid 25 can be stored in a receptable 18 such that the tube 36 aspirates it and dispenses to the penetration area before penetration. This is to prevent the cover liquid 25 from flowing out of the penetration area.

According to an embodiment, the apparatus 100 is programmable for enabling the syringe or pipette mechanism 84 to robotically: i) engage with a syringe 32 or a pipette 89 in the cartridge 10, and ii) disengage from the syringe 36 or the pipette 89 after placing in the cartridge 10. The apparatus 100 may comprise at least one heater module (not shown) for extraction of nucleic acid from the biological sample. The receiving module 102 is such that at least one receptacle 18 engages with the heater module for the extraction process. The syringe or pipette mechanism 84 engages with the syringe 36 or the pipette 89 in the cartridge module 10 to transfer out of the cartridge module 10 and disengages the syringe 36 or the pipette 89 after returning back to the cartridge module 10.

The sample container 115 may also have the cover layer 23 and the cover liquid 25 and may be transferrable to the cartridge module 10 for analyses. The syringe and tube pick up modules may return it back to the cartridge module 10 after the analyses.

The tube 36 preferably has a sharp tip for piercing the cover layer 23 with minimum damage in the cover layer 23. During the processes of aspiration and dispensation of the reagents 12 or the biological sample 21, at least one receptacle 18 within the cover layer 23 is maintained empty to act as an air reservoir for assisting in the air pressure regulation in other receptacles 18. An air filter 13 in cover layer 23 may be provided that blocks aerosol and spillage but allows air flow.

After the analyses, the cartridge module (10) with the receptacles (18) with the cover layer (23), reagent liquids (12), the biological sample (21), the tube (36), and the syringe (36) or the pipette (89) are disposed. Otherwise, any trace DNA left would be amplified and cause contamination.

According to an embodiment, the tube 36 used for the steps of nucleic acid extraction is wider than the tube 36 for the steps of PCR preparation before amplification. The wider tube 36 can accommodate magnetic beads in use and the protein molecules in the biological samples 21 without clogging and causing high resistance to their flow within the tube 36. The narrower tube 36 is used for the steps for PCR preparation for handling the reagents 12 and the biological sample 21 and loading into the receptacle 18 for amplification. The narrower tube 36 provides enough capillary force to avoid liquid dripping in air from the tube tip 37. The cartridge module 10 is provided with such tubes 36 and the tube pick-up module (not shown) in the apparatus 100 is capable of selecting the tube 36 that is appropriate for the step under consideration. The wider tube 36 may have an internal diameter of 0.5 mm to 3 mm and the narrower tube 36 may have an internal diameter of 0.1 mm to 0.45 mm. Other dimensions may also be used as required.

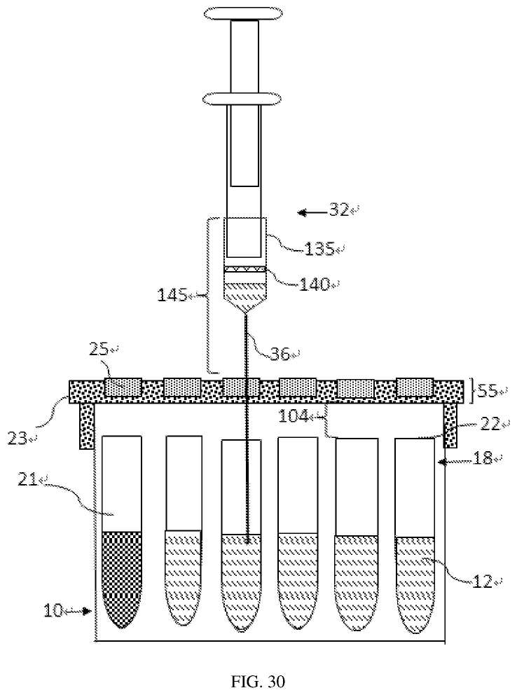

FIG. 30 is an elevation cross-sectional diagram of an embodiment, where the cartridge module 10 further comprises at least one tube holder 135 for holding the tube 36 and being engageable with the syringe 32. The tube holder 135 further comprises a tube filter 140, such that during aspiration and dispensation, the biological sample 21 and the reagent 12 are accommodated in the tube 36 and under the tube filter 140 and does not enter the syringe 32. The tube filter 140 allows passage of air and substantially blocks passage of aerosol or the reagent 12 and the biological sample 21 in use from reaching the syringe 32. This embodiment is helpful for disposing only the tube 36 along with the tube holder 135 and the filter 140 after use, and reusing the syringe 32 which is maintained substantially uncontaminated by the biological sample 21. This saves cost. The apparatus 100 can be operated to disengage the set 145 of the tube 36 along with the tube holder 135 and the filter 140 and attach a fresh set 145 to the syringe 32.

While replacing the tube 36, in order to minimize the aerosol contamination in the syringe 32 and the ambience, the method comprises the following steps: a) pushing down the plunger 66 to discharge aerosol contaminated air into the cartridge module 10, b) moving the syringe 32 with the tube 36 to a cleaner area; c) allowing the syringe 32 to aspirate air in the cleaner area; d) replacing the tube 32 by the apparatus 100 after following the steps at a) to c) in that order at least once, the tube (36) being attached to the syringe 32 or being a part of the set 145 comprising the tube 36, a tube holder 135 and a tube filter 140.

According to an embodiment, the cartridge module 10 may be provided with all the features as recited in the last claim.

The cover layer 23 may comprise an air passage filter 13 to facilitate the reduction in change of the air pressure, while substantially preventing the contamination due to spillage and blocking the aerosol exchange.

The method may further comprise: exempting at least one of the receptacles 18 from the aspiration and the dispensation, for further facilitating the reduction in change of air pressure by increasing the volume of the connecting air space 104.

What is claimed is:

1. A cartridge module for improving a contamination control during biological analyses or a nucleic acid analyses protocol comprising a nucleic acid extraction, a nucleic acid amplification, and a nucleic acid detection, the cartridge module comprising:
at least one hollow tube;
a plurality of receptacles; and
at least one cover layer to isolate the plurality of receptacles from an ambience outside the at least one cover layer, wherein each of the plurality of receptacles allows the at least one hollow tube, when attached to a syringe or a pipette in use, to dispense and aspirate an in use reagent liquid or in use biological sample to and from the plurality of receptacles through the at least one cover layer only from a plurality of receptacle top openings,
the at least one cover layer substantially blocks an aerosol exchange between the ambience and the plurality of receptacles during a whole process of the biological analyses or nucleic acid analyses even after the at least one hollow tube pierces the at least one cover layer and retracts,
the at least one cover layer also substantially prevents a contamination of the ambience due to a spillage of the in use reagent liquid or the in use biological sample from the plurality of receptacles or the cartridge module,
the at least one cover layer being attachable to the cartridge module or the plurality of receptacles while maintaining a connecting air space between the at least one cover layer and the plurality of the receptacle top openings together, the connecting air space being isolated from the ambience of the cartridge module by the at least one cover layer,
the connecting air space facilitating a reduction in a change of an air pressure inside isolated receptacles during an aspiration or a dispensation of the in use biological sample and the in use reagent liquid by the at least one hollow tube.

2. The cartridge module according to claim 1, wherein at least a portion of a cover layer material is elastomeric.

3. The cartridge module according to claim 1, further comprising:
a cover liquid pre-dispensed or provided to be dispensed at least one of being over or under or within the at least one cover layer at least where the at least one hollow tube pierces, to form a stack or be soaked by the at least one cover layer or be sandwiched within the at least one cover layer, the cover liquid being non-reacting and immiscible with the in use reagent liquid and the in use biological sample, the cover liquid being wetting over the at least one hollow tube and the at least one cover layer.

4. The cartridge module according to claim 3, further comprising:
a scrubbing layer fixed at least either of over and under the stack such that the at least one cover layer, the cover liquid, and the scrubbing layer form a composite layer, the scrubbing layer being pierceable by the at least one hollow tube and substantially scrubbing off an excess cover liquid sticking to the at least one hollow tube when being retracted out of the plurality of receptacles or when entering the plurality of receptacles respectively.

5. The cartridge module according to claim 1, further comprising the syringe, providing a cover liquid between a plunger and a barrel of the syringe,
the cover liquid substantially blocking an air communication between the ambience and the plurality of receptacles via the syringe, the cover liquid being immiscible and non-reacting with the in use reagent liquid and the in use biological sample.

6. The cartridge module according to claim 1, further comprising:
a rigid holding layer as positioned over or under the at least one cover layer, the rigid holding layer having holding layer apertures aligned over the plurality of receptacle top openings for the at least one hollow tube to pierce the at least one cover layer.

7. The cartridge module according to claim 1, further comprising:
at least one receptacle holder for holding at least one of the plurality of receptacles, the at least one receptacle holder being detachable from the cartridge module.

8. The cartridge module according to claim 1, further comprising:
a liquid sealant, wherein when the liquid sealant is dispensed over or under the at least one cover layer over at least one of the plurality of receptacle top openings, a cover layer aperture created in the at least one cover layer by a piercing of the at least one hollow tube is sealed whether with or without curing, the liquid sealant being immiscible and non-reacting with the in use reagent liquid and the in use biological sample.

9. The cartridge module according to claim 3, wherein the at least one cover layer is provided with pits to contain the cover liquid, and the pits are interconnected.

10. The cartridge module according to claim 1, wherein over the plurality of receptacle top openings, the at least one cover layer has a recessed top having slanted sides to provide a tapering towards a base region smaller than each of the plurality of receptacle top openings, the slanted sides being for guiding a tube tip of an in use hollow tube into the base region before the tube tip pierces the at least one cover layer through the base region.

11. The cartridge module according to claim 1, wherein at least a portion of the plurality of receptacles is made of a deformable material, so that in use,
(i) the plurality of receptacles in an initial condition or shape bloat to accommodate a dispensed reagent liquid or the in use biological sample, and
(ii) the plurality of receptacles in the initial condition or shape collapse when the dispensed reagent liquid or the in use biological sample is aspirated out,
thereby facilitating the reduction in the change of the air pressure inside the isolated receptacles during the aspiration or the dispensation of the in use biological sample and the in use reagent liquid by the at least one hollow tube.

12. The cartridge module according to claim 1, wherein the cartridge module further comprises: a sealing layer sealing the plurality of receptacle top openings individually or in plurality and under the connecting air space to prevent the spillage of the in use reagent liquid and the in use biological sample from the plurality of receptacles when the cartridge module is not in an upright position, the sealing layer being pierceable by the at least one hollow tube or peelable.

13. An apparatus for improving a contamination control during biological analyses or a nucleic acid analyses protocol comprising a nucleic acid extraction, a nucleic acid amplification, and a nucleic acid detection, the apparatus comprising:
a) at least one heater module for the nucleic acid extraction from the in use biological sample;
b) a receiving module for accommodating the cartridge module according to claim 1, such that at least one of the plurality of receptacles engages with the at least one heater module;
c) a robotically controlled syringe or pipette mechanism for operating the at least one hollow tube attached to the syringe or the pipette to aspirate and dispense the in use biological sample or the in use reagent liquid from and into the plurality of receptacles via the plurality of receptacle top openings and through the at least one cover layer or a stack or a sandwich, the robotically controlled syringe or pipette mechanism engaging to the syringe or the pipette in the cartridge module to transfer out of the cartridge module and disengaging the syringe or the pipette after returning back to the cartridge module;
d) an amplification module to provide an isothermal heating or a cyclic heating for the nucleic acid amplification in the in use biological sample within the at least one of the plurality of receptacles, the nucleic acid amplification being conductible while the at least one of the plurality of receptacles is within or outside the cartridge module;
e) an optical imaging mechanism for an imaging of the in use biological sample during or after the nucleic acid amplification, while the at least one of the plurality of receptacles containing the in use biological sample is within or outside the cartridge module; and
f) a robotically controlled transfer mechanism configurable to position the at least one of the plurality of receptacles as held by a receptacle holder as provided in the cartridge module or in the apparatus, to outside the cartridge module, when the nucleic acid amplification is conducted outside the cartridge module and returns the receptacle holder with the at least one of the plurality of receptacles to the cartridge module.

14. The apparatus according to claim 13, wherein the apparatus is computer programmable to operate the robotically controlled syringe or pipette mechanism to:
press the at least one hollow tube as attached to the syringe or the pipette against a fixed surface in the cartridge module or in the apparatus to bend the at least one hollow tube, and thereafter mount the syringe with the at least one hollow tube (36) in the cartridge module.

15. The apparatus according to claim 13, wherein the apparatus is further computer programmable to provide a substantially horizontal movement of the receptacle holder within the cartridge module such that during the aspiration and the dispensation of the in use reagent liquid and the in use biological sample by the robotically controlled syringe or pipette mechanism from and into the plurality of receptacles, a portion of the at least one hollow tube contacted the in use biological sample in the at least one of the plurality of receptacles does not move out of the connecting air space, during the substantially horizontal movement, all physical elements within the cartridge module remain within.

16. A method for improving a contamination control during biological analyses or a nucleic acid analyses protocol comprising a nucleic acid extraction, a nucleic acid amplification, and a nucleic acid detection, the method comprising:
employing the cartridge module and the apparatus according to claim 13;
loading the cartridge module with the in use reagent liquid and the in use biological sample in the plurality of receptacles into the receiving module;
conducting the biological analyses or a nucleic acid analysis within the apparatus without exposing the in use reagent liquid and the in use biological sample to the ambience, by aspirating and dispensing the in use reagent liquid and the in use biological sample from and into the plurality of receptacles by piercing and retracting the at least one hollow tube through the at least one cover layer;
at least once during analyses, cleaning the at least one hollow tube by aspirating and dispensing a cleaning liquid by the at least one hollow tube;
at least once, aspirating a filler liquid in the syringe by the at least one hollow tube; and
dispensing the filler liquid, such that the filler liquid;
a) fills a liquid retaining space in a tube head configured to otherwise retain the in use reagent liquid and the in use biological sample during a subsequent dispensation after the aspiration, or
b) replaces the in use reagent liquid and the in use biological sample pre-occupying the liquid retaining space from a previous processing,
the filler liquid being immiscible with, heavier than, and non-reacting with the in use reagent liquid and the in use biological sample; and
after the analyses, disposing the cartridge module with the plurality of receptacles with the at least one cover layer, the in use reagent liquids (12), the in use biological sample, the at least one hollow tube, and the syringe or the pipette.

17. The method according to claim 16, comprising:
employing the at least one hollow tube as attached to the pipette containing a filter and a liquid layer in the pipette, the filter and the liquid layer being separated by a first gap therebetween and the liquid layer being closer to a tube tip; and
operating the robotically controlled syringe or pipette mechanism to maintain a second gap between the liquid layer and the in use reagent liquid or the in use biological sample as aspirated.

18. The method according to claim 16, comprising:
adapting to either step selected from the group consisting of:
a) injecting a liquid sealant through the at least one cover layer to seal the plurality of receptacle top openings before an amplification of the in use biological sample in the at least one of the plurality of receptacles, and
b) disposing the liquid sealant on the at least one cover layer over the plurality of receptacle top openings or on the stack, before the amplification of the in use biological sample inside the at least one of the plurality of receptacles,
the liquid sealant being provided within the apparatus.

19. The method according to claim 16, comprising:
employing the cartridge module further comprising a sealing layer sealing the plurality of receptacle top openings individually or in plurality to be under the at least one cover layer to prevent the spillage of the in use reagent liquid and the in use biological sample from within the plurality of receptacles when the cartridge module is not in an upright position, the sealing layer being peelable or pierceable over the plurality of receptacle top openings by the at least one hollow tube; and
through the at least one cover layer as attached over the sealing layer, generating a vent aperture in the sealing layer over the plurality of receptacle top openings by the at least one hollow tube as attached to the robotically controlled syringe or pipette mechanism, or peeling off the sealing layer before attaching the at least one cover layer,
so that the at least one of the plurality of receptacles is in an air communication with the connecting air space during a subsequent aspiration or the dispensation from or into the at least one of the plurality of receptacles.

20. The method according to claim 16, further comprising:
conducting at least two steps of amplifications in the amplification module of the apparatus with a treatment of the in use biological sample by the in use reagent liquid between two consecutive applications and through the at least one cover layer or the stack, while the at least one of the plurality of receptacles containing the in use biological sample remains within the cartridge module or remains outside the cartridge module but within the apparatus.

21. The method according to claim 16, further comprising:
exempting the at least one of the plurality of receptacles from the aspiration and the dispensation, for further facilitating the reduction in the change of the air pressure by increasing a volume of the connecting air space.

22. The method according to claim 16, further comprising:
employing a first tube for the nucleic acid extraction to be wider than a second tube for polymerase chain reaction (PCR), such that the first tube accommodates magnetic beads in use and protein molecules in the in use biological sample without clogging and causing a high resistance and the second tube for PCR preparation steps like mixing of reagents to the in use biological sample and loading into the at least one of the plurality of receptacles for an amplification for an enough capillary force to avoid liquid dripping in air from a needle tip.

23. The method according to claim 22, wherein:
the first tube has an internal diameter of 0.5 mm to 3 mm and the second tube has an internal diameter of 0.1 mm to 0.45 mm.

24. The method according to claim 16, further comprising:
while replacing the at least one hollow tube, reducing an aerosol contamination in the syringe and the ambience by the following steps:
a) pushing down a plunger to discharge an aerosol contaminated air into the cartridge module,
b) moving the syringe with the at least one hollow tube to a cleaner area;
c) allowing the syringe (32) to aspirate aerosol contaminated air in the cleaner area;
d) replacing the at least one hollow tube by the apparatus after following the steps at a) to c) in order at least once,
the at least one hollow tube being attached to the syringe or being a part of a set comprising the at least one hollow tube, a tube holder, and a tube filter.

25. The cartridge module according to claim 7, wherein the cartridge module allows a substantially horizontal movement of the at least one receptacle holder within the cartridge module such that during the substantially horizontal movement, all physical elements within the cartridge module remain within the cartridge module.

26. The cartridge module according to claim 1, wherein the at least one cover layer comprises an air passage filter to facilitate the reduction in the change of the air pressure, while substantially preventing the contamination due to spillage and blocking the aerosol exchange.

27. The cartridge module according to claim 1, further comprising:
at least one tube holder for holding the at least one hollow tube and being engageable with the syringe; and
a tube filter in the at least one tube holder, such that during the aspiration and the dispensation, the in use biological sample and the in use reagent liquid are accommodated in the at least one hollow tube and under the tube filter,
the tube filter allowing a passage of air and substantially blocking a passage of an aerosol or the in use reagent liquid and the in use biological sample from reaching the syringe.

28. A cartridge module for improving a contamination control during biological analyses or a nucleic acid analyses protocol comprising a nucleic acid extraction, a nucleic acid amplification, and a nucleic acid detection, the cartridge module comprising:
at least one hollow tube;
a plurality of receptacles; and
at least one cover layer to isolate the plurality of receptacles from an ambience outside the at least one cover layer, wherein each of the plurality of receptacles allows the at least one hollow tube, when attached to a syringe or a pipette in use, to dispense and aspirate an in use reagent liquid or an in use biological sample to and from each of the plurality of receptacles through the at least one cover layer only from a plurality of receptacle top openings,
the at least one cover layer substantially blocks an aerosol exchange between the ambience and each of the plurality of receptacles during a whole process of the biological analyses or a nucleic acid analyses even after the at least one hollow tube pierces the at least one cover layer and retracts,
the at least one cover layer also substantially prevents a contamination of the ambience due to a spillage of the in use reagent liquid or the in use biological sample from the plurality of receptacles or the cartridge module,
the at least one cover layer being attachable to the cartridge module or the plurality of receptacles while maintaining a connecting air space between the at least one cover layer and the plurality of the receptacle top openings together, the connecting air space being isolated from the ambience of the cartridge module by the at least one cover layer,
the connecting air space facilitating a reduction in a change of an air pressure inside isolated receptacles during an aspiration or a dispensation of the in use biological sample and the in use reagent liquid by the at least one hollow tube;
wherein a cover layer material is elastomeric at least where the at least one hollow tube pierces the at least one cover layer and retracts;
a cover liquid pre-dispensed or provided to be dispensed at least either of on and under the at least one cover layer at least where the at least one hollow tube pierces, to form a stack or be soaked by the at least one cover layer or be sandwiched within the at least one cover layer, the cover liquid being non-reacting and immiscible with the in use reagent liquid and the in use biological sample, the cover liquid being wetting over the at least one hollow tube and the at least one cover layer;
a scrubbing layer fixed at least either of over and under the stack such that the at least one cover layer, the cover liquid, and the scrubbing layer form a composite layer, the scrubbing layer being pierceable by the at least one hollow tube and substantially scrubbing off an excess cover liquid sticking to the at least one hollow tube when being retracted out of the plurality of receptacles;
the syringe, providing the cover liquid between a plunger and a barrel of the syringe, the cover liquid substantially blocking an air communication between the ambience and the plurality of receptacles via the syringe, the cover liquid being immiscible and non-reacting with the in use reagent liquid and the in use biological sample;
a rigid holding layer as positioned over or under the at least one cover layer, the rigid holding layer having holding layer apertures aligned over the plurality of receptacle top openings for the at least one hollow tube to pierce the at least one cover layer;
at least one receptacle holder for holding at least one of the plurality of receptacles with the at least one cover layer, the at least one receptacle holder being detachable from the cartridge module;
a liquid sealant, wherein when the liquid sealant is dispensed over or under the at least one cover layer over at least one of the plurality of receptacle top openings, a cover layer aperture created in the at least one cover layer by a piercing of the at least one hollow tube is sealed whether with or without curing, the liquid sealant being immiscible and non-reacting with the in use reagent liquid and the in use biological sample;
the at least one cover layer is provided with pits to contain the cover liquid, and the pits are interconnected;
over the plurality of receptacle top openings, the at least one cover layer has a recessed top having slanted sides to provide a tapering towards a base region smaller than each of the plurality of receptacle top openings, the slanted sides being for guiding a tube tip of an in use hollow tube into the base region before the tube tip pierces the at least one cover layer through the base region;
a non-elastomeric sealing layer sealing the plurality of receptacle top openings individually or in plurality and under the connecting air space to prevent the spillage of the in use reagent liquid and the in use biological sample from the plurality of receptacles when the cartridge module is not in an upright position, the sealing layer being pierceable by the at least one hollow tube or peelable, and
at least one tube holder for holding the at least one hollow tube and being engageable with the syringe; and
a tube filter in the at least one tube holder, such that during the aspiration and the dispensation, the in use biological sample and the in use reagent liquid are accommodated in the at least one hollow tube and under the tube filter, the tube filter allowing a passage of air and substantially blocking a passage of an aerosol or the in use reagent liquid and the in use biological sample from reaching the syringe.
